(19) United States
(12) Patent Application Publication
(10) Pub. No.: US 2023/0365608 A1
(43) Pub. Date: Nov. 16, 2023

(54) ANTIVIRAL NUCLEOSIDES AND DERIVATIVES THEREOF

(71) Applicant: Janssen BioPharma, Inc., South San Francisco, CA (US)

(72) Inventors: Leonid BEIGELMAN, San Mateo, CA (US); Guangyi WANG, Irvine, CA (US); Natalia DYATKINA, Mountain View, CA (US)

(21) Appl. No.: 17/426,974

(22) PCT Filed: Jan. 30, 2020
(86) PCT No.: PCT/IB2020/050747

§ 371 Date: Jul. 29, 2021

Related U.S. Application Data
(60) Provisional application No. 62/800,124, filed on Feb. 1, 2019.

(51) Int. Cl. C07H 7/06; C07H 15/04; A61P 31/16

(52) U.S. Cl. CPC C07H 7/06; C07H 15/04; A61P 31/16

(57) ABSTRACT

Disclosed herein are nucleoside compounds and derivatives thereof, pharmaceutical compositions containing same, and their methods of synthesis. The compounds are useful in treating orthomyxovirus infections, such as influenza infections.

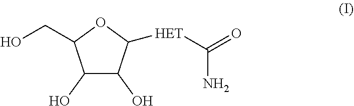

### BACKGROUND OF THE INVENTION

### Field of the Invention

The present invention relates to nucleoside compounds and derivatives thereof, pharmaceutical compositions comprising these compounds, processes for preparing same and their use in treating an orthomyxovirus or influenza infection in animals, particularly humans.

### Description

The viruses of the Orthomyxoviridae family are negative-sense, single-stranded RNA viruses. The Orthomyxoviridae family contains several genera including Influenza virus A, Influenza virus B, Influenza virus C, Isavirus and Thogotovirus. Influenza viruses can cause respiratory viral infections, including upper and lower respiratory tract viral infections. Respiratory viral infections are a leading cause of death of millions of people each year. Upper respiratory tract viral infections involve the nose, sinuses, pharynx and/or larynx. Lower respiratory tract viral infections involve the respiratory system below the vocal cords, including the trachea, primary bronchi and lungs.

Influenza is a negative sense, single stranded RNA virus and a member of the Orthomyxoviridae family. There are currently three species of influenza; influenza A, influenza B and influenza C. Influenza A has a lipid membrane derived from the host cell, which contains the hemagglutinin, neuraminidase and M2 proteins that project from the surface of the virus. Influenza A has been further classified based on two viral surface proteins, namely hemagglutinin (H or HA) and neuraminidase (N). There are approximately 16 Hantigens (H1 to H16) and 9 N antigens (N1 to N9). Influenza A includes several subtypes, including H1N1, H1N2, H2N2, H3NI, H3N2, H3N8, H5N1, H5N2, H5N3, H5N8, H5N9, H7N1, H7N2, H7N3, H7N4, H7N7, H7N9, H9N2 and H10N7. The influenza virus polymerase is a heterotrimer composed of three subunits, polymerase acid (PA), polymerase basic 1 (PB1) and polymerase basic 2 (PB2). This polymerase is responsible for replication and transcription of the viral RNA in the nuclei of infected cells. The PA subunit contains the endonuclease active site. The endonuclease activity of the PA cleaves the cellular mRNA, which is then used by the PB1 subunit as a primer for the viral mRNA synthesis.

Influenza viruses can be transmitted from person to person via direct contact with infected secretions and/or contaminated surfaces or objections. Complications from an influenza viral infection include pneumonia, bronchitis, dehydration, and sinus and ear infections. In an effort to combat the toll of influenza infection, many countries have adopted vaccinations as a preventive measure with limited success due to the difficulty of predicting which influenza strain(s) will dominate in the impending flu season. Even when the correct strain(s) are identified, vaccinations offer incomplete immunity against the flu as evidenced by the spike in hospitalizations and deaths for the 2017-2018 flu season. Universal vaccines capable of protecting against all flu strains have not yet been developed despite active research in this area. Medications currently approved by the FDA against influenza infections include a limited number of neuraminidase inhibitors and M2 protein inhibitors. Examples of approved neuraminidase inhibitors and M2 protein inhibitors include amantadine, rimantadine, Relenza® (zanamivir, GlaxoSmithKline) and Tamiflu® (oseltamivir, Genentech). To date, there are no therapeutic compounds targeting the influenza polymerase complex available on the market in the United States. Hence, there is a need for compounds which can address the disadvantages or limitations of current approaches.

### SUMMARY OF THE INVENTION

The invention is directed to the general and preferred embodiments defined, respectively, by the independent and dependent claims appended hereto, which are incorporated by reference herein. One aspect of this invention concerns compounds of Formula (I):

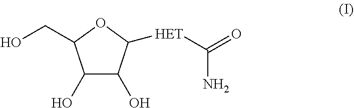

wherein
HET is a heteroaryl selected from the group consisting of:

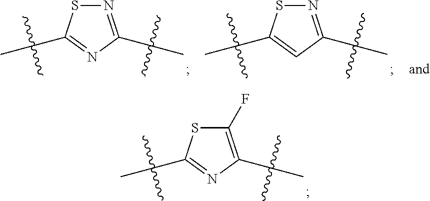 [SMILES: *c1nsc(*)n1 ; *c1cc(*)sn1 ; *c1nc(*)c(F)s1]

and pharmaceutically acceptable salts, solvates, stereoisomers, isotopic variants, or N-oxides of compounds of Formula (I).

Further embodiments are provided by pharmaceutically acceptable salts of compounds of Formula (I) (as well as Formula (II), Formula (III), and Formula (IV)), pharmaceutically acceptable prodrugs of compounds of Formula (I) (as well as Formula (II), Formula (III), and Formula (IV)), and pharmaceutically active metabolites of compounds of Formula (I) (as well as Formula (II), Formula (III), and Formula (IV)).

In certain embodiments, the compounds of Formula (I) are compounds selected from those species described or exemplified in the detailed description below.

In a further aspect, the invention relates to enantiomers and diastereomers of the compounds of Formula (I) (as well as Formula (II), Formula (III), and Formula (IV)), as well as the pharmaceutically acceptable salts.

In a further aspect, the invention relates to pharmaceutical compositions for treating an orthomyxovirus viral infection, comprising an effective amount of at least one compound selected from compounds of Formula (I) (as well as Formula (II), Formula (III), and Formula (IV)), pharmaceutically acceptable salts of compounds of Formula (I) (as well as Formula (II), Formula (III), and Formula (IV)), pharmaceutically acceptable prodrugs of compounds of Formula (I) (as well as Formula (II), Formula (III), and Formula (IV)), and pharmaceutically active metabolites of Formula (I) (as well as Formula (II), Formula (III), and Formula (IV)).

Pharmaceutical compositions according to the invention may further comprise one or more pharmaceutically acceptable excipients.

In another aspect, the invention is directed to a method of treating a subject suffering from, or diagnosed with an orthomyxovirus viral infection, comprising administering to the subject in need of such treatment an effective amount of at least one compound selected from compounds of Formula (I) (as well as Formula (II), Formula (III), and Formula (IV)), pharmaceutically acceptable salts of compounds of Formula (I) (as well as Formula (II), Formula (III), and Formula (IV)), pharmaceutically acceptable prodrugs of compounds of Formula (I) (as well as Formula (II), Formula (III), and Formula (IV)), and pharmaceutically active metabolites of compounds of Formula (I) (as well as Formula (II), Formula (III), and Formula (IV)). Additional embodiments of methods of treatment are set forth in the detailed description.

Additional embodiments of this invention include methods of making compounds of Formula (I) (as well as Formula (II), Formula (III), and Formula (IV)), pharmaceutically acceptable salts of compounds of Formula (I), (as well as Formula (II), Formula (III), and Formula (IV)) pharmaceutically acceptable prodrugs of compounds of Formula (I) (as well as Formula (II), Formula (III), and Formula (IV)), and pharmaceutically active metabolites of Formula (I) (as well as Formula (II), Formula (III), and Formula (IV)).

An object of the present invention is to overcome or ameliorate at least one of the disadvantages of the conventional methodologies and/or prior art, or to provide a useful alternative thereto.

Additional embodiments, features, and advantages of the invention will be apparent from the following detailed description and through practice of the invention.

The invention may be more fully appreciated by reference to the following description, including the following glossary of terms and the concluding examples. For the sake of brevity, the disclosures of the publications, including patents, cited in this specification are herein incorporated by reference.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, the terms “including”, “containing” and “comprising” are used herein in their open, non-limiting sense.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₆ alkyl” or similar designations. By way of example only, “C₁-C₆ alkyl” indicates that there are one to six carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl (straight and branched) and hexyl (straight and branched). The alkyl group may be substituted or unsubstituted.

The term “heteroaryl” or “HET” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

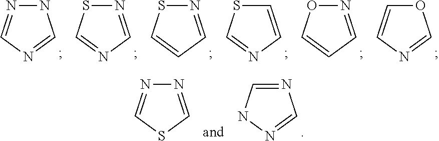

Those skilled in the art will recognize that the species of heteroaryl groups listed or illustrated above are not exhaustive, and that additional species within the scope of these defined terms may also be selected.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972).

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl and isobutyryl); arylalkylcarbonyls and arylalkoxycarbonyls (e.g., benzyloxycarbonyl); substituted methyl ether (e.g. methoxymethyl ether and tetrahydropyranyl ether); substituted ethyl ether; a substituted benzyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl and t-butyldiphenylsilyl); esters (e.g. benzoate ester); carbonates (e.g. methoxymethylcarbonate); sulfonates (e.g. tosylate and mesylate); acyclic ketal (e.g. dimethyl acetal and diisopropyl acetal); cyclic ketals (e.g., 1,3-dioxane and 1,3-dioxolane); acyclic acetal; cyclic acetal; acyclic hemiacetal; cyclic hemiacetal; dithioacetals (both cyclic and acyclic); dithioketals (both cyclic and acyclic) (e.g., S,S′-dimethyl, S,S′-diethyl, S,S′-diispropyl, 1,3-dithiane and 1,3-dithiolane); orthoesters (including cyclic orthoesters, such as cyclic orthoformates); carbamates (e.g., N-phenylcarbamate) and triarylmethyl groups (e.g., trityl, monomethoxytrityl (MMTr), 4,4′-dimethoxytrityl (DMTr), and 4,4′,4″-trimethoxytrityl (TMTr); and those described herein). “Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates, mesylates, trifluoroacetates and halogens (e.g., I, Br, and Cl). Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference for the limited purpose of disclosing characteristics and examples of leaving groups.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid.

Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Compounds

In one aspect, provided herein are compounds of Formula (I), and pharmaceutically acceptable salts, stereoisomers, isotopic variants, N-oxides, or solvates thereof,

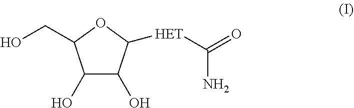

wherein
HET is a heteroaryl selected from the group consisting of:

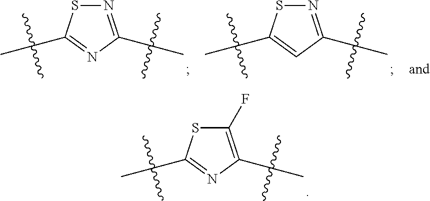 [SMILES: *c1nsc(*)n1 ; *c1cc(*)sn1 ; *c1nc(*)c(F)s1]

An additional embodiment of the invention is a compound of Formula (I) wherein HET is

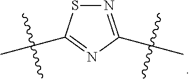 [SMILES: *c1nsc(*)n1]

An additional embodiment of the invention is a compound of Formula (I) wherein HET is

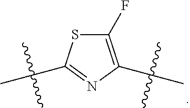 [SMILES: *c1nc(*)c(F)s1]

An additional embodiment of the invention is a compound of Formula (I) wherein HET is

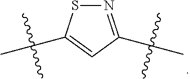 [SMILES: *c1cc(*)sn1]

A further embodiment of the current invention is a compound selected from the group consisting of:

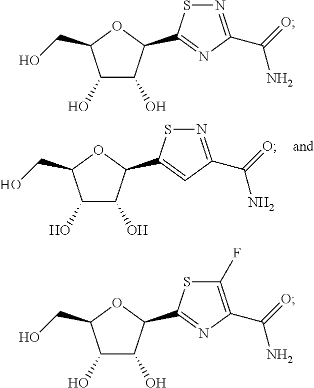

and pharmaceutically acceptable salts, solvates, or N-oxides thereof.

A further embodiment of the current invention is a compound having the structure:

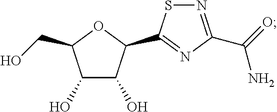 [SMILES: NC(=O)c1nsc([C@@H]2O[C@H](CO)[C@@H](O)[C@H]2O)n1]

and pharmaceutically acceptable salts, solvates, or N-oxides thereof.

An additional embodiment of the invention is a compound of Formula (II), and pharmaceutically acceptable salts, stereoisomers, isotopic variants, N-oxides, or solvates thereof,

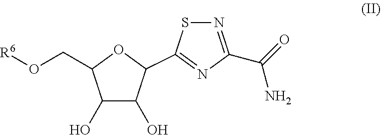

wherein
R⁶ is —(C═O)C₁₋₆alkyl, or —(C═O)C₁₋₆alkyl wherein C₁₋₆alkyl is substituted with NH₂.

A further embodiment of the current invention is a compound selected from the group consisting of:

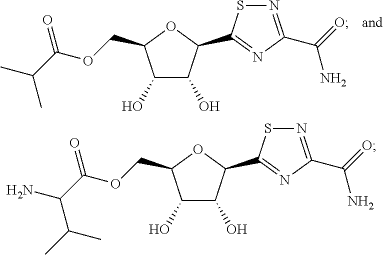

and pharmaceutically acceptable salts, solvates, or N-oxides thereof.

An additional embodiment of the invention is a compound of Formula (III), and pharmaceutically acceptable salts, stereoisomers, isotopic variants, N-oxides, or solvates thereof,

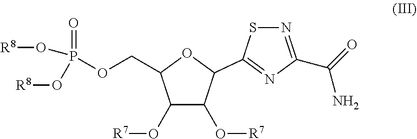

wherein
R⁷ is H or two R⁷ members come together to form a 5-membered ring substituted with OCH₃; and
R⁸ is —CH₂O—(C═O)—O—C₁₋₆alkyl.

A further embodiment of the current invention is a compound selected from the group consisting of:

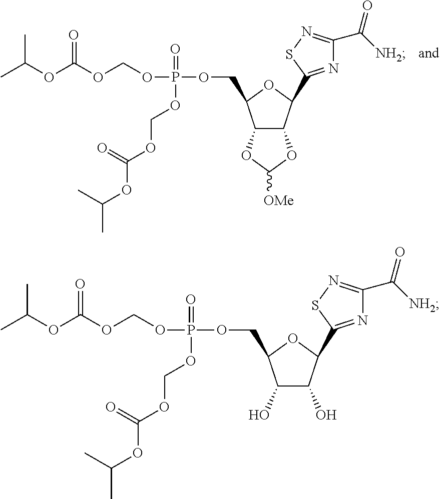

and pharmaceutically acceptable salts, solvates, or N-oxides thereof.

An additional embodiment of the invention is a compound of Formula (IV), and pharmaceutically acceptable salts, stereoisomers, isotopic variants, N-oxides, or solvates thereof,

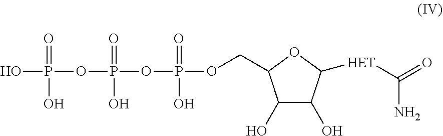

wherein
HET is a heteroaryl selected from the group consisting of:

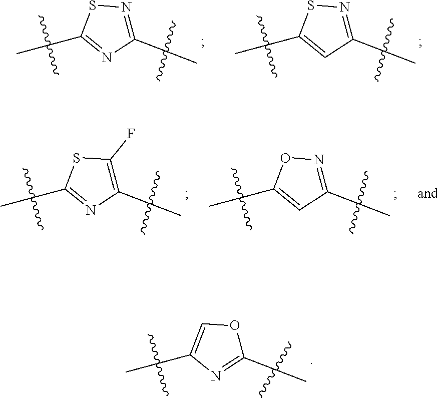 [SMILES: *c1nsc(*)n1 ; *c1cc(*)sn1 ; *c1nc(*)c(F)s1 ; *c1cc(*)on1 ; *c1coc(*)n1]

A further embodiment of the current invention is a compound selected from the group consisting of:

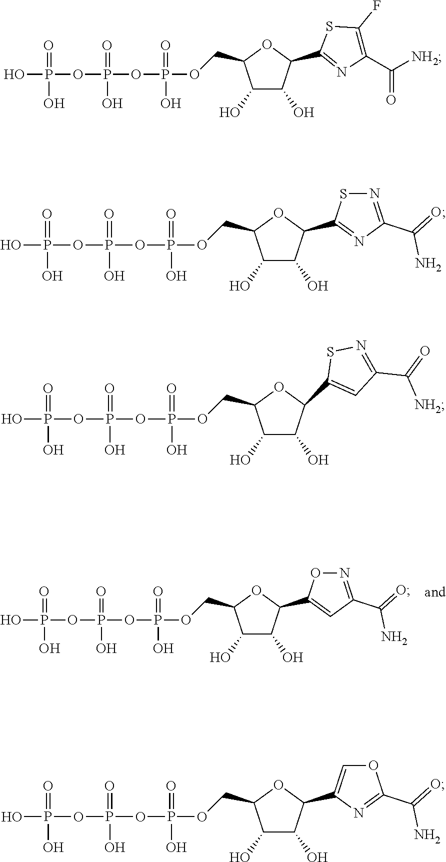 [SMILES: NC(=O)c1nc([C@@H]2O[C@H](COP(=O)(O)OP(=O)(O)OP(=O)(O)O)[C@@H](O)[C@H]2O)sc1F ; NC(=O)c1nsc([C@@H]2O[C@H](COP(=O)(O)OP(=O)(O)OP(=O)(O)O)[C@@H](O)[C@H]2O)n1 ; NC(=O)c1cc([C@@H]2O[C@H](COP(=O)(O)OP(=O)(O)OP(=O)(O)O)[C@@H](O)[C@H]2O)sn1 ; NC(=O)c1cc([C@@H]2O[C@H](COP(=O)(O)OP(=O)(O)OP(=O)(O)O)[C@@H](O)[C@H]2O)on1 ; NC(=O)c1nc([C@@H]2O[C@H](COP(=O)(O)OP(=O)(O)OP(=O)(O)O)[C@@H](O)[C@H]2O)co1]

and pharmaceutically acceptable salts, solvates, or N-oxides thereof.

A further embodiment of the current invention is a compound as shown below in Table 1.

| Example # | Structure |
|---|---|
| 1 | 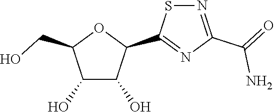 [SMILES: NC(=O)c1nsc([C@@H]2O[C@H](CO)[C@@H](O)[C@H]2O)n1] |
| 2 | 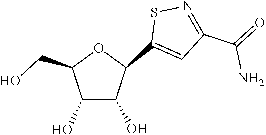 [SMILES: NC(=O)c1cc([C@@H]2O[C@H](CO)[C@@H](O)[C@H]2O)sn1] |
| 3 | 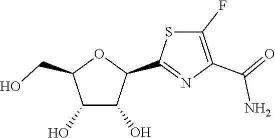 [SMILES: NC(=O)c1nc([C@@H]2O[C@H](CO)[C@@H](O)[C@H]2O)sc1F] |
| 4 | 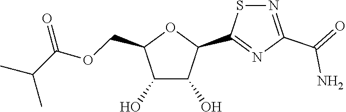 [SMILES: CC(C)C(=O)OC[C@H]1O[C@@H](c2nc(C(N)=O)ns2)[C@H](O)[C@@H]1O] |
| 6 | 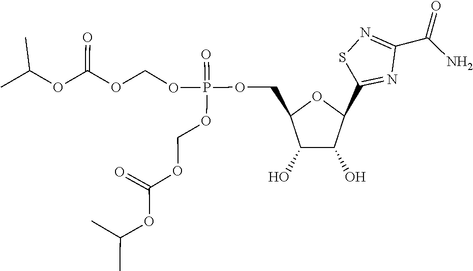 [SMILES: CC(C)OC(=O)OCOP(=O)(OCOC(=O)OC(C)C)OC[C@H]1O[C@@H](c2nc(C(N)=O)ns2)[C@H](O)[C@@H]1O] |
| 7 | 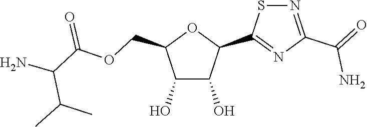 [SMILES: CC(C)C(N)C(=O)OC[C@H]1O[C@@H](c2nc(C(N)=O)ns2)[C@H](O)[C@@H]1O] |

and pharmaceutically acceptable salts, N-oxides, or solvates thereof.

A further embodiment of the current invention is a compound as shown below in Table 2.

| Example # | Structure |
|---|---|
| 9 | 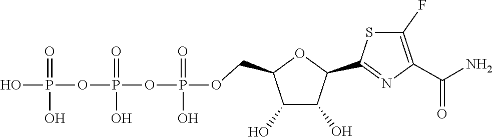 [SMILES: NC(=O)c1nc([C@@H]2O[C@H](COP(=O)(O)OP(=O)(O)OP(=O)(O)O)[C@@H](O)[C@H]2O)sc1F] |
| 10 | 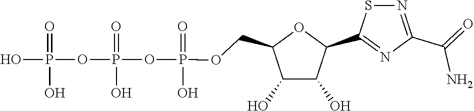 [SMILES: NC(=O)c1nsc([C@@H]2O[C@H](COP(=O)(O)OP(=O)(O)OP(=O)(O)O)[C@@H](O)[C@H]2O)n1] |
| 11 | 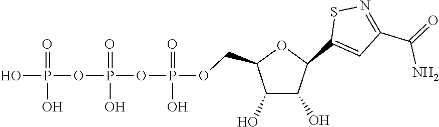 [SMILES: NC(=O)c1cc([C@@H]2O[C@H](COP(=O)(O)OP(=O)(O)OP(=O)(O)O)[C@@H](O)[C@H]2O)sn1] |
| 12 | 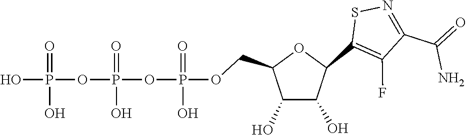 [SMILES: NC(=O)c1nsc([C@@H]2O[C@H](COP(=O)(O)OP(=O)(O)OP(=O)(O)O)[C@@H](O)[C@H]2O)c1F] |
| 13 | 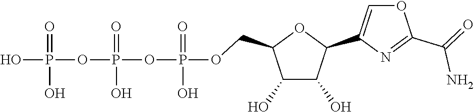 [SMILES: NC(=O)c1nc([C@@H]2O[C@H](COP(=O)(O)OP(=O)(O)OP(=O)(O)O)[C@@H](O)[C@H]2O)co1] |

and pharmaceutically acceptable salts, N-oxides, or solvates thereof.

Pharmaceutical Compositions

Some embodiments described herein relate to a pharmaceutical composition, that can include an effective amount of one or more compounds described herein (e.g., a compound of Formula (I) (as well as Formulas (II), (III) and (IV)), or a pharmaceutically acceptable salt thereof) and a pharmaceutically acceptable carrier, diluent, excipient or combination thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. Suitable excipients can be found in the Handbook of Pharmaceutical Excipients, published by the American Pharmaceutical Association, which is incorporated herein by reference. A “diluent” is a type of excipient.

The pharmaceutical compositions described herein can be administered to a human patient perse, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be administering intramuscular. In other embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be administering intranasal. In still other embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be administering intradermal. In yet still other embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be administering orally.

When administered orally, one or more compounds described herein (e.g., a compound of Formula (I) (as well as Formulas (II), (III) and (IV)), or a pharmaceutically acceptable salt thereof) can be formulated as tablets, pills, dragees, capsules, liquids, gels, syrups, slurries, suspensions and the like, for oral ingestion by a subject to be treated. Injectables can be prepared in conventional forms, either as liquid solutions or suspensions, solid forms suitable for solution or suspension in liquid prior to injection, or as emulsions. Pharmaceutical compositions for intranasal delivery may also include drops and sprays often prepared to assist in simulating nasal secretions.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

An additional embodiment of the invention is a pharmaceutical composition comprising and effective amount of at least one compound in Table 1, as well as pharmaceutically acceptable salts, N-oxides or solvates of compounds of Table 1, pharmaceutically acceptable prodrugs of compounds of Table 1, and pharmaceutically active metabolites of Table 1; and at least one pharmaceutically acceptable excipient.

An additional embodiment of the invention is a pharmaceutical composition comprising and effective amount of at least one compound in Table 2, as well as pharmaceutically acceptable salts, N-oxides or solvates of compounds of Table 2, pharmaceutically acceptable prodrugs of compounds of Table 2, and pharmaceutically active metabolites of Table 2; and at least one pharmaceutically acceptable excipient.

Also within the scope of the invention are enantiomers and diastereomers of the compounds of Formula (I) (as well as Formulas (II), (Ill), and (IV)). Also within the scope of the invention are the pharmaceutically acceptable salts, N-oxides or solvates of the compounds of Formula (I) (as well as Formulas (II), (Ill), and (IV)). Also within the scope of the invention are the pharmaceutically acceptable prodrugs of compounds of Formula (I) (as well as Formulas (II), (Ill), and (IV)), and pharmaceutically active metabolites of the compounds of Formula (I) (as well as Formulas (II), (Ill), and (IV)).

Also within the scope of the invention are isotopic variations of compounds of Formula (I) (as well as Formulas (II), (Ill), and (IV)), such as, e.g., deuterated compounds of Formula (I). Also within the scope of the invention are the pharmaceutically acceptable salts, N-oxides or solvates of the isotopic variations of the compounds of Formula (I) (as well as Formulas (II), (Ill), and (IV)). Also within the scope of the invention are the pharmaceutically acceptable prodrugs of the isotopic variations of the compounds of Formula (I) (as well as Formulas (II), (Ill), and (IV)), and pharmaceutically active metabolites of the isotopic variations of the compounds of Formula (I) (as well as Formulas (II), (Ill), and (IV)). Compounds of the present invention can be provided in the form of a prodrug, namely a compound which is metabolized in vivo to the active metabolite. Conventional procedures for the selection and preparation of suitable prodrug derivatives are described, for example, in “Design of Prodrugs”, ed. H. Bundgaard, Elsevier, 1985.

### Methods of Use:

Some embodiments described herein relate to a method of ameliorating and/or treating an orthomyxovirus infection, e.g. influenza virus infection, which can include administering an effective amount of one or more compounds described herein, or a pharmaceutical composition that includes one or more compounds described herein (e.g., a compound of Formula (I), or a pharmaceutically acceptable salt thereof).

Other embodiments described herein relate to a method of inhibiting an orthomyxovirus viral replication, e.g. influenza viral replication, which can include contacting a cell infected with the orthomyxovirus virus, e.g. influenza viral replication, with an effective amount of a compound of Formula (I), or a pharmaceutically acceptable salt thereof and/or a pharmaceutical composition that includes one or more compounds described herein (e.g., a compound of Formula (I), or a pharmaceutically acceptable salt thereof).

In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used to treat and/or ameliorate an influenza viral infection. In other embodiments, an effective amount of one or more compounds of Formula (I), or a pharmaceutically acceptable salt thereof and/or a pharmaceutical composition that includes one or more compounds described herein (e.g., a compound of Formula (I), or a pharmaceutically acceptable salt thereof) can be used to

In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used to inhibit the replication an influenza virus. In some embodiments, an effective amount of one or more compounds of Formula (I), or a pharmaceutically acceptable salt thereof and/or a pharmaceutical composition that includes one or more compounds described herein (e.g., a compound of Formula (I), or a pharmaceutically acceptable salt thereof) can be used to inhibit activity of the influenza polymerase complex. In some embodiments, an effective amount of one or more compounds of Formula (I), or a pharmaceutically acceptable salt thereof and/or a pharmaceutical composition that includes one or more compounds described herein (e.g., a compound of Formula (I), or a pharmaceutically acceptable salt thereof) can be used for inhibiting and/or reducing the endonuclease activity of an influenza endonuclease that can include contacting the active site of the endonuclease with a compound of Formula (I), or a pharmaceutically acceptable salt thereof. In some embodiments, one or more compounds described herein inhibits and/or reduces the ability of the endonuclease to cleave the mRNA.

In some embodiments, including those embodiments in the previous paragraphs, the influenza viral infection can be an influenza A viral infection. In other embodiments, including those embodiments in the previous paragraphs, the influenza viral infection can be an influenza B viral infection. In still other embodiments, including those embodiments in the previous paragraphs, the influenza viral infection can be an influenza C viral infection. In some embodiments, a compound of Formula (I), or a pharmaceutically acceptable salt thereof, can be used to treat and/or ameliorate one or more subtypes of influenza. For example, a compound of Formula (I), or a pharmaceutically acceptable salt thereof, can be used to treat H1N1 and/or H3N2. In addition, or in the alternative, a compound of Formula (I), or a pharmaceutically acceptable salt thereof, can be used to treat H2N2, H5N1 and/or H7N9. In some embodiments, a compound described herein (a compound of Formula (I), or a pharmaceutically acceptable salt thereof) can be effective against more than 1 subtype of influenza. For example, a compound described herein (a compound of Formula (I), or a pharmaceutically acceptable salt thereof can be effective against 2, 3, 4, and/or 5 or more subtypes of influenza.

In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used treat and/or ameliorate an upper respiratory viral infection attributed to (directly and/or indirectly) an influenza virus infection. In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used treat and/or ameliorate a lower respiratory viral infection attributed to (directly and/or indirectly) an influenza virus infection. In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used treat and/or ameliorate one or more symptoms of an influenza virus infection (such as those described herein). In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used treat and/or ameliorate bronchiolitis and/or tracheobronchitis due to an influenza virus infection. In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used treat and/or ameliorate pneumonia due to an influenza virus infection. In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used treat and/or ameliorate coup due to an influenza virus infection.

In some embodiments, an effective amount of one or more compounds of Formula (I), or a pharmaceutically acceptable salt thereof, and/or a pharmaceutical composition that includes one or more compounds described herein (e.g., a compound of Formula (I), or a pharmaceutically acceptable salt thereof) can be used to lessen the severity of one or more symptoms of an influenza infection; examples of symptoms include, but are not limited to: fever, chills, cough, sore throat, runny nose, stuffy nose, muscle aches, body aches, headache, fatigue, vomiting and/or diarrhea.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy.

The terms “therapeutically effective amount” and “effective amount” are used to indicate an amount of an active compound, or pharmaceutical agent, that elicits the biological or medicinal response indicated. For example, a therapeutically effective amount of compound can be the amount needed to alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The therapeutically effective amount of the compounds disclosed herein required as a dose will depend on the route of administration, the type of animal, including human, being treated, and the condition or physical characteristics of the specific animal under consideration. The dose can be tailored to achieve a desired effect, but will depend on such factors as age, weight, diet, concurrent medication and other factors which those skilled in the medical arts will recognize.

As used herein, the term “subject” refers to an animal, preferably a mammal, most preferably a human, who has been the object of treatment or observation. “Mammal” includes, without limitation, mice, rats, rabbits, guinea pigs, dogs, cats, sheep, goats, cows, horses, primates, such as monkeys, chimpanzees, and apes, and humans. In some embodiments, the subject is human.

Various indicators for determining the effectiveness of a method for treating an orthomyxovirus viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, ameliorate symptoms of the disease, and/or other indicator of disease response.

In some embodiments, a compound of Formula (I), or a pharmaceutically acceptable salt of the foregoing, can result in one or more improvement in quality of life, such as reduced illness duration, reduced illness severity, reduced time to return to normal health and normal activity, and reduced time to alleviation of one or more symptoms of orthomyxovirus infection, compared to a subject who is untreated. In other embodiments, a compound of Formula (I), or a pharmaceutically acceptable salt of the foregoing, can result in one or more improvement in quality of life, such as reduced illness duration, reduced illness severity, reduced time to return to normal health and normal activity, and reduced time to alleviation of one or more symptoms of orthomyxovirus infection, compared to a subject receiving conventional standard of care for treating influenza. In some embodiments, a compound of Formula (I), or a pharmaceutically acceptable salt of the foregoing, can result in a reduced length and/or severity of one or more symptoms associated with an orthomyxovirus infection compared to an untreated subject. Symptoms of an orthomyxovirus infection are described herein and include but not limited to chills, cough, myalgia (muscle pain), nasal obstruction, sore throat, fatigue, headache and fever. In some embodiments, a compound of Formula (I), or a pharmaceutically acceptable salt of the thereof, can result in a reduction in one or more secondary complications associated with an orthomyxovirus infection, including but not limited to otitis media (ear inflammation), sinusitis, bronchitis and pneumonia compared to an untreated subject.

In some embodiments, a compound of Formula (I), or a pharmaceutically acceptable salt of the foregoing, can result in at least a 1, 2, 3, 4, 5, 10, 15, 20, 25, 50, 75, 100-fold or more reduction in the replication of an orthomyxovirus relative to pre-treatment levels in a subject, as determined after initiation of the treatment regime (for example, 10 days after initiation of treatment). In some embodiments, a compound of Formula (I), or a pharmaceutically acceptable salt of the foregoing, can result in a reduction of the replication of an orthomyxovirus relative to pre-treatment levels in the range of about 2 to about 5 fold, about 10 to about 20 fold, about 15 to about 40 fold, or about 50 to about 100 fold. In some embodiments, a compound of Formula (I), or a pharmaceutically acceptable salt thereof, can result in a reduction of orthomyxovirus replication in the range of 1 to 1.5 log, 1.5 log to 2 log, 2 log to 2.5 log, 2.5 to 3 log, or 3 to 3.5 log reduction of orthomyxovirus replication compared to the reduction of orthomyxovirus reduction achieved by oseltamivir (Tamiflu®), or may achieve the same reduction as that of oseltamivir (Tamiflu®) therapy in a shorter period of time, for example, in one day, two days, three days, or four days as compared to the reduction achieved after 5 days of oseltamivir (Tamiflu®) therapy.

After a period of time, infectious agents can develop resistance to one or more select therapeutic agents. According to the CDC, many influenza A strains have developed resistance to the class of influenza drugs known as adamantanes, which include amantadine and rimantadine. Likewise, strains of H1N1 influenza viruses are known to possess resistance to oseltamivir. The term “resistance” as used herein refers to a viral strain displaying a delayed, lessened and/or null response to a therapeutic agent(s). For example, after treatment with an antiviral agent, the viral load of a subject infected with a resistant virus may be reduced to a lesser degree compared to the amount in viral load reduction exhibited by a subject infected with a non-resistant strain. In some embodiments, a compound of Formula (I) (as well as Formulas (II), (III) and (IV)), or a pharmaceutically acceptable salt thereof, can be administered to a subject infected with a strain of influenza virus that is resistant to one or more different anti-influenza agents (for example, amantadine, rimantadine and/or oseltamivir). In some embodiments, a compound of Formula (I), or a pharmaceutically acceptable salt thereof, can be administered to a subject infected with an influenza virus that is resistant to a M2 protein inhibitor.

In some embodiments, a compound of Formula (I), or a pharmaceutically acceptable salt thereof, can decrease the percentage of subjects that experience complications from an influenza viral infection compared to the percentage of subjects that experience complication being treated with oseltamivir. For example, the percentage of subjects being treated with a compound of Formula (I), or a pharmaceutically acceptable salt thereof, that experience complications can be 10%, 25%, 40%, 50%, 60%, 70%, 80% and 90% less compared to subjects being treated with oseltamivir.

In some embodiments, a compound of Formula (I), or a pharmaceutically acceptable salt thereof, or a pharmaceutical composition that includes a compound described herein, can be used in combination with one or more additional agent(s). In some embodiments, a compound of Formula (I), or a pharmaceutically acceptable salt thereof, can be used in combination with one or more agents currently used in a conventional standard of care for treating influenza. For example, the additional agent can be amantadine (adamantan-1-amine, Symmetrel®), rimantadine (Flumadine®), zanamivir (Relenza®) and oseltamivir (Tamiflu®). For the treatment of influenza, additional agents include but are not limited to a neuraminidase inhibitor, a M2 protein inhibitor, a polymerase inhibitor, a PB2 inhibitor, peramivir ((1S,2S,3S,4R)-3-[(1S)-1-acetamido-2-ethylbutyl]-4-(diaminomethylideneamino)-2-hydroxycyclopentane-1-carboxylic acid, BioCryst Pharmaceuticals), Ianinamivir ((4S,5R,6R)-5-acetamido-4-carbamimidamido-6-[(1R,2R)-3-hydroxy-2-methoxypropyl]-5,6-dihydro-4H-pyran-2-carboxylic acid), favipiravir (T-705, 6-fluoro-3-hydroxy-2-pyrazinecarboxamide), Ianinamivir octanoate ((3R,4S)-3-acetamido-4-guanidino-2-((1S,2S)-2-hydroxy-1-methoxy-3-(octanoyloxy)propyl)-3,4-dihydro-2H-pyran-6-carboxylic acid) fludase (DAS181, NexBio), ADS-8902 (amantadine HCl/oseltamivir/ribavirin, Adamas Pharmaceuticals), an immuno-modulator (for example, a Type 1 interferon), beraprost (4-[2-hydroxy-1-[(E)-3-hydroxy-4-methyloct-1-en-6-ynyl]-2,3,3a,8b-tetrahydro-1H-cyclopenta[b][1]benzofuran-5-yl]butanoic acid), Neugene®, ribavirin, (R)-3-((5-fluoro-2-(5-fluoro-1H-pyrrolo[2,3-b]pyridin-3-yl)pyrimidin-4-yl)amino)-4,4-dimethylpentanoic acid (CAS Reg. No. 1422050-75-6), (2S,3S)-3-((5-fluoro-2-(5-fluoro-1H-pyrrolo[2,3-b]pyridin-3-yl)pyrimidin-4-yl)amino)bicyclo[2.2.2]octane-2-carboxylic acid (CAS Reg. No. 1259366-34-1, VX-787), (S)-8-benzhydryl-4-hydroxy-6-isopropyl-7,8-dihydro-3H-pyrazino[1,2-b]pyridazine-3,5 (6H)-dione, (S)-8-benzhydryl-6-isopropyl-3,5-dioxo-5,6,7,8-tetrahydro-3H-pyrazino[1,2-b]pyridazin-4-yl isobutyrate FluMist Quadrivalent® (MedImmune), Fluarix® Quadrivalent (GlaxoSmithKline), Fluzone® Quadrivalent (Sanofi Pasteur), Flucelvax® (Novartis) and FluBlok® (Protein Sciences). In some embodiments, a compound of Formula (I), or a pharmaceutically acceptable salt thereof, or a pharmaceutical composition that includes a compound described herein, can be used in combination with oseltamivir.

Type 1 interferons are known to those skilled in the art. A non-limiting list of examples include: alpha-interferons, beta-interferons, delta-interferons, omega-interferons, tau-interferons, x-interferons, consensus interferons and asialo-interferons. Type 1 interferons can be pegylated. Examples of specific type 1 interferons include interferon alpha 1A, interferon alpha 1B, interferon alpha 2A, interferon alpha 2B, pegylated-interferon alpha 2a (PEGASYS, Roche), recombinant interferon alpha 2a (ROFERON, Roche), inhaled interferon alpha 2b (AERX, Aradigm), pegylated-interferon alpha 2b (ALBUFERON, Human Genome Sciences/Novartis, PEGINTRON, Schering), recombinant interferon alpha 2b (INTRON A, Schering), pegylated interferon alpha 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical).

In some embodiments, a compound of Formula (I), or a pharmaceutically acceptable salt thereof, can be administered with one or more additional agent(s) together in a single pharmaceutical composition. In some embodiments, a compound of Formula (I), or a pharmaceutically acceptable salt thereof, can be administered with one or more additional agent(s) as two or more separate pharmaceutical compositions. For example, a compound of Formula (I), or a pharmaceutically acceptable salt thereof, can be administered in one pharmaceutical composition, and at least one of the additional agents can be administered in a second pharmaceutical composition. If there are at least two additional agents, one or more of the additional agents can be in a first pharmaceutical composition that includes a compound of Formula (I), or a pharmaceutically acceptable salt thereof, and at least one of the other additional agent(s) can be in a second pharmaceutical composition.

The order of administration of a compound of Formula (I), or a pharmaceutically acceptable salt thereof, with one or more additional agent(s) can vary. In some embodiments, a compound of Formula (I), or a pharmaceutically acceptable salt thereof, can be administered prior to all additional agents. In other embodiments, a compound of Formula (I), or a pharmaceutically acceptable salt thereof, can be administered prior to at least one additional agent. In still other embodiments, a compound of Formula (I), or a pharmaceutically acceptable salt thereof, can be administered concomitantly with one or more additional agent(s). In yet still other embodiments, a compound of Formula (I), or a pharmaceutically acceptable salt thereof, can be administered subsequent to the administration of at least one additional agent. In some embodiments, a compound of Formula (I), or a pharmaceutically acceptable salt thereof, can be administered subsequent to the administration of all additional agents.

The route of administration, exact dosage and frequency of administration depends on the particular compound of formula (I) (or formula (II), (Ill), or (IV)) used, the mammalian species treated, the particular condition being treated, the severity of the condition being treated, the age, weight and general physical condition of the particular patient as well as other medication the individual may be taking, as is well known to those skilled in the art. Furthermore, it is evident that said effective amount may be lowered or increased depending on the response of the treated subject and/or depending on the evaluation of the physician prescribing the compounds of the instant invention. The effective daily amount ranges mentioned hereinabove are therefore only guidelines and are not intended to limit the scope or use of the invention to any extent. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

### Synthesis

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations and acronyms used herein include the following:

TABLE 2

| Term | Acronym |
|---|---|
| Acetonitrile | ACN |
| Aqueous | aq |
| Atmosphere | atm |
| Benzotriazol-1-yloxy-tris(dimethylamino) | BOP |
| phosphonium hexafluorophosphate | |
| Broad | br |
| Diatomaceous Earth | Celite ® |
| 1,8-Diazabicyclo[5.4.0]undec-7-ene | DBU |
| N,N′-Dicyclohexylcarbodiimide | DCC |
| Dichloromethane | DCM |
| Diisopropylethylamine | DIPEA, DIEA, |
| | or Hunig's base |
| 4-Dimethylaminopyridine | DMAP |
| N,N-Dimethylformamide | DMF |
| Dimethylsulfoxide | DMSO |
| 1-Ethyl-3-(3-dimethylaminopropyl)carbodiimide | EDCI, EDAC, |
| | or EDC |
| Diethyl ether | Ether, Et₂O |
| Ethyl Acetate | EtOAc, or EA |
| Ethanol | EtOH |
| Normal-phase silica gel chromatography | FCC |
| Grams | g |
| Hours | h |
| High-pressure liquid chromatography | HPLC |
| Hertz | Hz |
| Isopropyl alcohol | iPrOH, IPA |
| Liquid chromatography and mass spectrometry | LCMS |
| Molar | M |
| Mass to charge ratio | m/z |
| meta-Chloroperoxybenzoic acid | mCPBA |
| Methanol | MeOH |
| Milligrams | mg |
| Minute | min |
| Milliliter | mL |
| Microliter | μL |
| Millimoles | mmol |
| Mass spectrometry | MS |
| Normal | N |
| N-Bromosuccinimide | NBS |
| N-Chlorosuccinimide | NCS |
| N-lodosuccinimide | NIS |
| Nuclear magnetic resonance | NMR |
| CF₃SO₃— or triflate | OTf |
| Parts per million | ppm |
| Precipitate | ppt |
| Retention time | Rₜ |
| Room temperature | rt |
| Saturated | sat |
| 1-Chloromethyl-4-fluoro-1,4-diazoniabicyclo | Select-Fluor ® |
| [2.2.2]octane bis(tetrafluoroborate) | |
| [2-(Trimethylsilyl)ethoxy]methyl acetal | SEM |
| Supercritical Fluid Chromatography | SFC |
| Temperature | T |
| Triethylamine | TEA |
| Trifluoroacetic acid | TFA |
| Tetrahydrofuran | THF |
| Thin layer chromatography | TLC |
| Volume in milliliters of solvent per gram of substrate | V, or volumes |

### Preparative Examples

Exemplary compounds useful in methods of the invention will now be described by reference to the illustrative synthetic schemes for their general preparation below and the specific examples to follow.

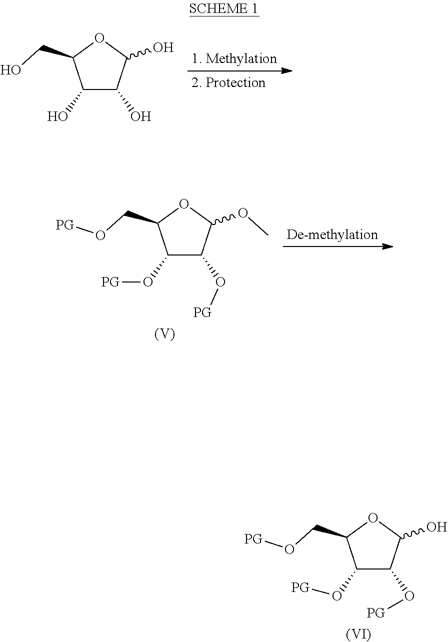

According to Scheme 1, a compound of formula (V), where PG is benzyl, is prepared from D-ribofuranose in two steps. In a first step, D-ribofuranose is methylated employing an acid such as H₂SO₄, in MeOH. In a second step, protection with a suitable protecting group such as benzyl, employing conditions known to one skilled in the art, provides a compound of formula (V). Removal of the methyl group in a compound of formula (V) is accomplished using an acid such a TFA, and the like, in water, for a period of 10-15 h, to provide a compound of formula (VI).

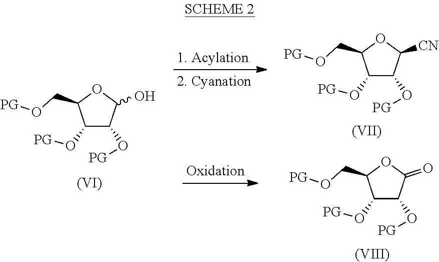

According to SCHEME 2, a compound of formula (VI), where PG is benzyl, is acetylated and subsequently treated with TMSCN/BF₃OEt₂ to give a ribofuranosyl cyanide compound of formula (VII) and its epimer. A compound of formula (VI) is also oxidized, employing oxidation conditions such as PCC, and the like, in a suitable solvent such as DCM, to provide a compound of formula (VIII).

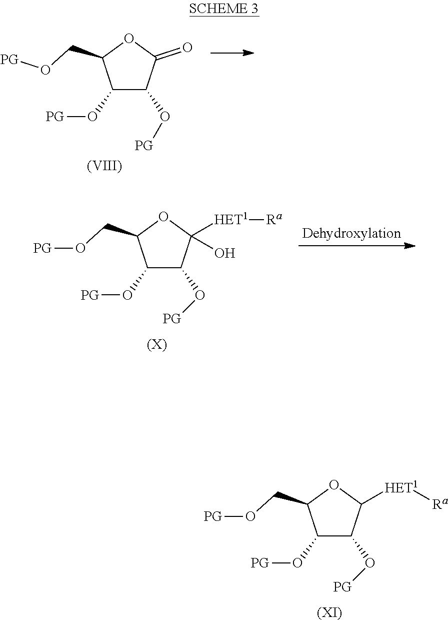

According to SCHEME 3, a ribolactone compound of formula (VIII) is reacted with 3-((4-methoxybenzyl)oxy)isothiazole, in the presence of a base such as LDA, and the like, in a suitable solvent such as Et₂O, and the like, at −78° C., to provide a compound of formula (X), where HET is isothiazole, and R^{a} is -OPMB. Dehydroxylation of a compound of formula (X) employing Et₃SiH and BF₃·Et₂O, in a suitable solvent such as DCM, and the like, at a temperature ranging from −78° C., to 25° C., affords a compound of formula (XI), where R^{a} is —OH or -SMe. It will be understood that the 4-methoxybenzyl protecting group (PMB) is removed under the dehydroxylation conditions as described above.

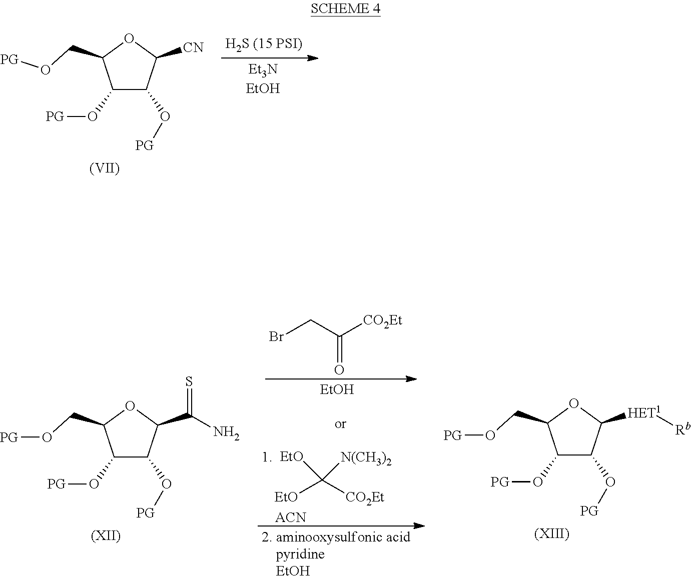

According to SCHEME 4, a compound of formula (VII), where PG is benzyl, is converted a ribofuranosyl thioamide compound of formula (XII), under pressure with H₂S, a suitable base such as Et₃N, pyridine, and the like, in a suitable solvent such as EtOH, and the like.

A ribofuranosyl thioamide compound of formula (XII) is cyclocondensed in a Hantzsch reaction, with ethyl ethyl 3-bromo-2-oxo-propanoate, in a suitable solvent such as EtOH, t-BuOH, and the like, employing conventional or microwave heating, to provide a thiazole compound of formula (XIII), where R^{b} is CO₂Et, and PG is benzyl.

A thiadiazole compound of formula (XIII), where R^{b} is CO₂Et, and PG is benzyl is synthesized in two steps from a ribofuranosyl thioamide compound of formula (XII), where PG is benzyl. In a first step, a ribofuranosyl thioamide compound of formula (XII) is reacted with commercially available or synthetically accessible ethyl 2-(dimethylamino)-2,2-diethoxyacetate (Intermediate 1), in a solvent such as ACN, and the like, employing microwave or conventional heating. In a second subsequent step, cyclization of the substituted carbothioamide in the presence of aminooxysulfonic acid (HAOS), a base such as pyridine, in a solvent such as EtOH, and the like, at a temperature of about 55° C., provides a thiadiazole compound of formula (XIII), where R^{b} is CO₂Et, and PG is benzyl.

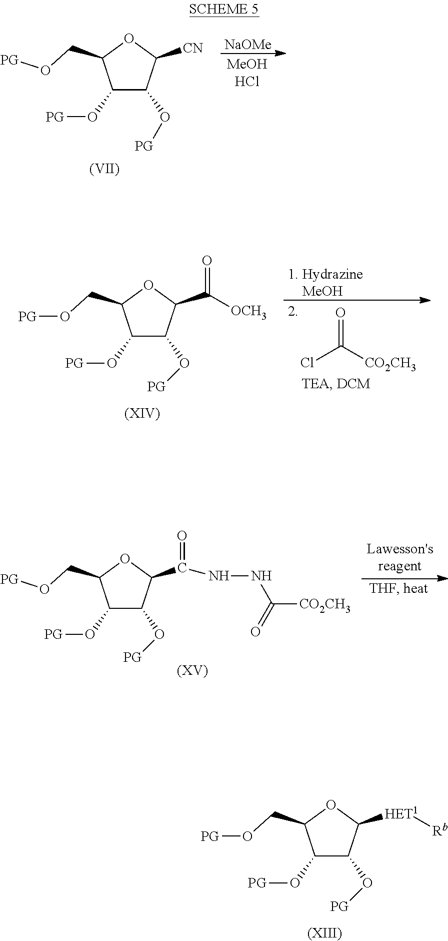

According to SCHEME 5, a compound of formula (VII), where PG is benzyl, is reacted with sodium methoxide, subsequent hydrolysis of the resulting imidate in situ with HCl provides a methyl ester compound of formula (XIV). A compound of formula (XV) is prepared in two steps from a compound of formula (XIV). In a first step, reaction with hydrazine, to provide the hydrazide intermediate, which in turn was acetylated with methyl 2-chloro-2-oxoacetate affords the substituted hydrazide of formula (XV). A compound of formula (XIII), where HET¹ is thiadiazole, and R^{b} is —CO₂CH₃, is formed in a one-pot thiolation and condensation of a compound of formula (XV) using Lawesson's reagent at an elevated temperature.

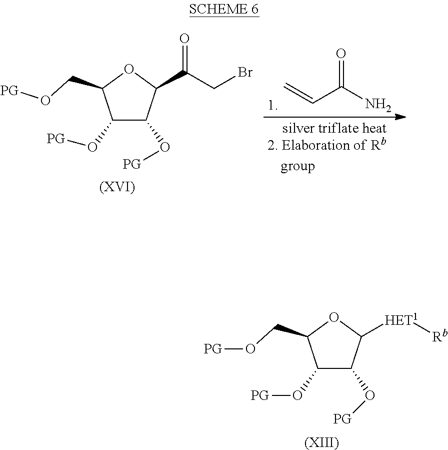

According to SCHEME 6, a commercially available or synthetically accessible compound of formula (XVI), where PG is benzyl, is reacted with acrylamide, in the presence of silver triflate, at a temperature of about 70° C., to provide a compound of formula (XVIII), where HET is oxazole, and R^{b} is —CH═CH₂. Elaboration of the R^{b} vinyl to R^{b} is —C(═O)NH₂ is achieved in 4 steps. In a first step, oxidation of the vinyl R^{b} moiety is achieved employing a catalyst such as OsO₄, an oxidant such as NMO, in a suitable solvent such as THF, acetone, water, or a mixture thereof, to provide a diol compound where R^{b} is CH(OH)CH₂OH. A diol compound is oxidatively cleaved with sodium periodate, to provide a compound where R^{b} is —C(═O)H. Oxidation of an aldehyde compound, employing conditions known to one skilled in the art, provides a compound where R^{b} is —CO₂H. A compound of formula (XIII), where R^{b} is —C(═O)NH₂ is obtained from a compound where R^{b} is —CO₂H, by reaction with an amine under amide bond formation conditions. In a preferred embodiment, the amine is ammonia, is reacted with a compound where R^{b} is —CO₂H, in the presence of a dehydrating agent such as HOBt/EDAC, CDI, HATU, HOAT, BOP; in an organic solvent or mixture thereof, such as toluene, acetonitrile, ethyl acetate, DMF, THF, methylene chloride, and the like; to afford a compound of formula (XIII), where R^{b} is —C(═O)NH₂. In a particularly preferred embodiment the dehydrating agent is HATU.

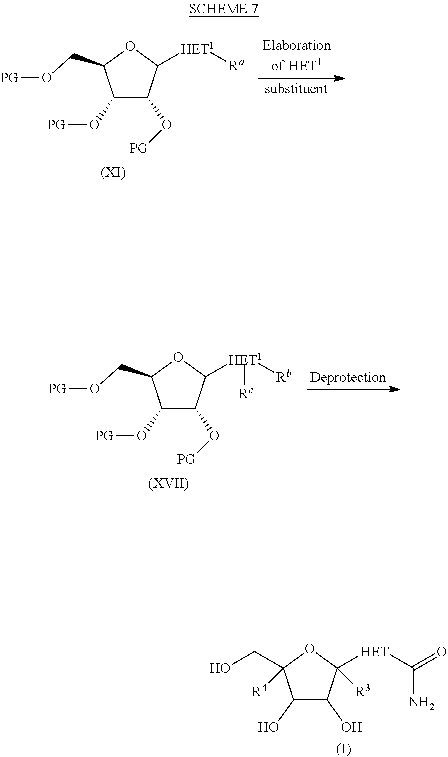

According to SCHEME 7, A compound of formula (XI), where R^{a} is -SMe, is oxidized employing conditions known to one skilled in the art. For example, reaction of a compound of formula (XI), where R^{a} is -SMe, with an oxidizing agent such as meta-chloroperoxybenzoic acid (mCPBA), in a suitable solvent such as DMC, at a temperature ranging from 0° C. to 25° C., to provide a compound of formula (XVII), where R^{b} is —SO₂Me and R^{c} is H. Conversion of the —SO₂Me to CN is achieved using KCN, NaCN, and the like, in a suitable solvent such as DMSO, to provide a compound of formula (XVII) where R^{b} is —CN and R^{c} is H.

A compound of formula (XVII), where R^{b} is CN, and R^{c} is H is hydrolyzed to a compound of formula (XVII) where R^{b} is —CO₂H, under basic conditions. For example, reaction of a compound of formula (XVII), where R^{b} is CN, and R^{c} is H is hydrolyzed to a compound of formula (XVII) where R^{b} is —CO₂H, with a base such as KOH in a suitable solvent mixture such as MeOH, H₂O, and THF, at a temperature of about 90° C., for a period of 18-24 h, to provide a compound of formula (XVII) where R^{b} is —CO₂H. Esterification of a compound of formula (XVII) where R^{b} is —CO₂H, is achieved using an alcohol such as 2-methylpropan-2-ol, and the like, DMAP, DCC, in a solvent such as DCM, to provide a compound of formula (XVII) where R^{b} is —CO₂C₁₋₄alkyl. A compound of formula (XIII) or (XVII), where R^{b} is —CO₂C₁₋₄alkyl is reacted with a base such as LDA, at a temperature of about −78° C., and a trialkyl tin reagent such as trimethyltin chloride, tributylchlorostannane, and the like, to provide a compound of formula (XVII) where R^{b} is —CO₂C₁₋₄alkyl, and R^{c} is Sn(C₁₋₄alkyl)₃. A compound of formula (XVII) where R^{b} is —CO₂C₁₋₄alkyl, and R^{c} is Sn(C₁₋₄alkyl)₃, is fluorinated in a silver-mediated fluorination reaction. For example, a compound of formula (XVII) where R^{b} is —CO₂C₁₋₄alkyl, and R^{c} is Sn(C₁₋₄alkyl)₃, is reacted with a suitable silver reagent such as Ag₂O, AgOTf, or a mixture thereof, a fluorinating agent such as Selectfluor®, a base such as NaOH, K₂CO₃, NaHCO₃, and the like, in a suitable solvent such as acetone, EtOAc, and the like, at a temperature of about 65° C., to provide a compound of formula (XVII), where R^{b} is —CO₂C₁₋₄alkyl, and R^{c} is F. Direct transformation of the ester functionality of a compound of formula (XIII) or (XVII), where R^{b} is —CO₂C₁₋₄alkyl, and R^{c} is H or F, to an amide compound of formula (XVII), where R^{b} is —C(═O)NH₂, and R^{c} is H or F, is achieved using NH₃-MeOH, at a temperature of about 50° C., for a period of 18-24 h.

Conversion of a compound of formula (XVII), where R^{b} is CN, and R^{c} is H or Cl, to a compound of formula (XVII), where R^{b} is —C(═O)NH₂, is achieved under alkaline conditions in the presence of hydrogen peroxide. For example, reaction of a compound of formula (XVII), where R^{b} is CN, with a base such as NH₃—H₂O, and the like, and H₂O₂, at room temperature, provides a compound of formula (XVII), where R^{b} is —C(═O)NH₂ and R^{c} is H or Cl.

A compound of formula (XI), (XIII), or (XVII), where PG is benzyl, is deprotected employing BCl₃, in a suitable solvent such as DCM, at temperatures ranging from −78° C. to 0° C., provides a compound of Formula (I), where HET is

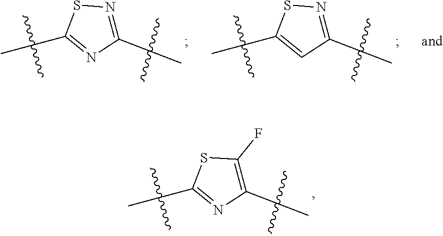 [SMILES: *c1nsc(*)n1 ; *c1cc(*)sn1 ; *c1nc(*)c(F)s1]

and R³ and R⁴ are H.

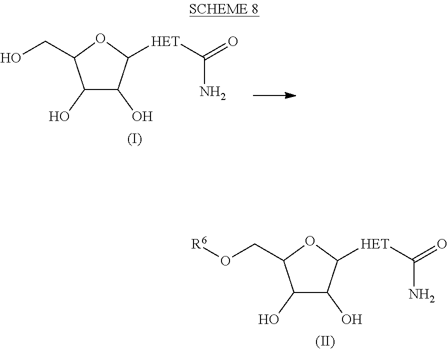

According to SCHEME 8, a compound of Formula (I) is acylated to provide a compound of Formula (II). In a first step, the secondary hydroxyl groups of a compound of Formula (I), are protected as oxomethylene-tethered, by treatment with trimethyl orthoformate and a catalytic amount of p-toluenesulfonic acid monohydrate. In a second step, acylated with an acid chloride such as propionyl chloride, isobutyryl chloride, BzCl, and the like, a base such as pyridine, and the like, and a catalyst such as DMAP, in a suitable solvent such as DCM. A protected as oxomethylene-tethered compound is alternately reacted with Boc-valine, and the like, and DCC, in a suitable solvent such as DMF.

Deprotection of the tethered oxomethylene is accomplished employing an acid such as HCl, in a suitable solvent such as dioxane, water, or a mixture thereof, provides a compound of Formula (II), where R⁶ is —C(═O)C₁₋₆alkyl or —C(═O)CH(NH₂)C₁₋₆alkyl.

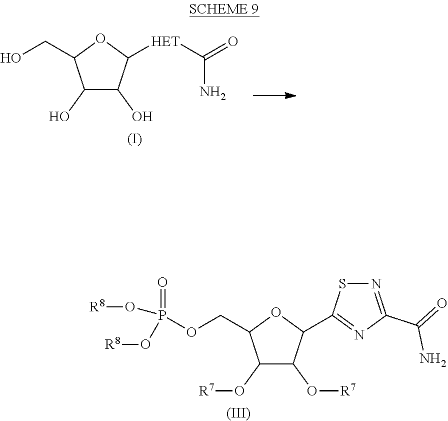

According to SCHEME 9, a compound of Formula (I), where HET is thiadiazole, is protected as oxomethylene-tethered, by treatment with trimethyl orthoformate and a catalytic amount of p-toluenesulfonic acid monohydrate. The oxomethylene-tethered compound is reacted in a second step with triethylammonium bis(POC)phosphate, a base such as DIPEA, and the like, BopCl, and nitrotriazole, in a suitable solvent such as THF, and the like, into provide a compound of Formula (III), where two R⁷ members come together to form a 5-membered ring substituted with OCH₃, and R⁸ is —CH₂O—(C═O)—O—C₁₋₆alkyl.

Deprotection of the tethered oxomethylene is accomplished employing an acid such as HCl, in a suitable solvent such as dioxane, water, or a mixture thereof, provides a compound of Formula (II), where R⁷ is H and R⁸ is —CH₂O—(C═O)—O—C₁₋₆alkyl.

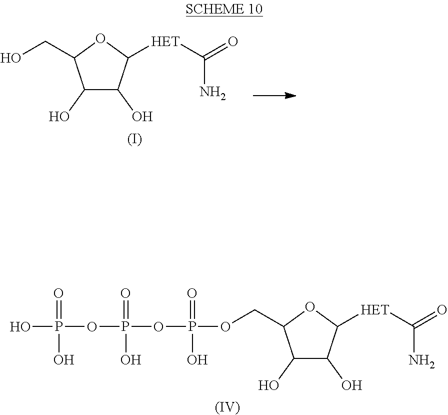

According to SCHEME 10, a nucleoside compound of Formula (IV), where HET

is

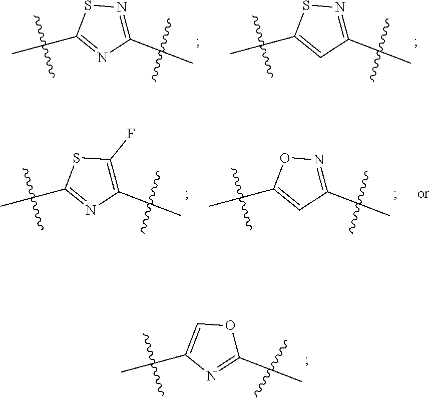 [SMILES: *c1nsc(*)n1 ; *c1cc(*)sn1 ; *c1nc(*)c(F)s1 ; *c1cc(*)on1 ; *c1coc(*)n1]

prepared from a compound of Formula (I), employing conditions known to one skilled in the art. For example, reaction of the nucleoside of Formula (I), with trimethyl phosphate, phosphoryl chloride, and N-methylimidazole to provide the monophosphate. Subsequent reaction with the tetrabutylammonium salt of pyrophosphate, in a suitable solvent such as DMF, and the like, provides the triphosphate of Formula (IV).

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### EXAMPLES

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were concentrated on a rotary evaporator under reduced pressure. Reactions under microwave irradiation conditions were carried out in a Biotage Initiator.

Normal-phase silica gel chromatography (FCC) was performed on silica gel (SiO₂) using prepacked cartridges.

Preparative reverse-phase high performance liquid chromatography (RP HPLC) was performed on:

An Agilent HPLC with an Xterra Prep RP18 column (5 μM, 30×100 or 50×150 mm) or an XBridge C18 OBD column (5 μM, 30×100 or 50×150 mm), and a mobile phase of 5% ACN in 20 mM NH₄HCO₃ was held for 2 min, then a gradient of 5-99% ACN over 15 min, then held at 99% ACN for 5 min, with a flow rate of 40 or 80 mL/min.

Mass spectra (MS) were obtained on an Agilent series 1100 MSD using electrospray ionization (ESI) in positive mode. Mass spectra of NTPs were obtained in negative mode. Calculated (calcd.) mass corresponds to the exact mass.

Nuclear magnetic resonance (NMR) spectra were obtained on Bruker model DRX spectrometers or Varian 400. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, MA) or ACD/Name Version 10.01 (Advanced Chemistry).

### Intermediate 1: Ethyl 2-(dimethylamino)-2,2-diethoxyacetate

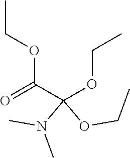

Step A: Ethyl 2-(dimethylamino)-2-oxoacetate. To a solution of ethyl 2-chloro-2-oxoacetate (100.00 g, 732.45 mmol, 81.95 mL) in DCM (2.0 L) was added Et₃N (133.4 g, 1.32 mol, 182.75 mL), then added N-methylmethanamine hydrochloride (107.5 g, 1.32 mol, 1.80 eq.) drop-wise at 0° C. The mixture was stirred at 25° C. for 2 h. The reaction was quenched by addition of EtOH (100 mL), and concentrated at low pressure. The residue was purified by column chromatography (FCC, SiO₂, PE/EA=5/1) to give the title compound (93.00 g, 634.27 mmol, 86.60% yield, 99% purity) as yellow oil.

¹H NMR (400 MHz, CDCl₃) δ=4.32 (q, J=7.3 Hz, 2H), 3.01 (s, 3H), 2.97 (s, 3H), 1.35 (t, J=7.2 Hz, 3H). LCMS: MS: m/z 145.9 [M+H]⁺.

Step B. Ethyl 2-(dimethylamino)-2,2-diethoxyacetate Ethyl 2-(dimethylamino)-2-oxoacetate (90 g, 620 mmol, 1.00 eq.) was treated with triethyloxonium tetrafluoroborate (117.8 g, 620 mmol) and the mixture was refluxed for 1 h at 100° C. The above mixture was cooled down to room temperature and treated with a solution of NaOEt (prepared by Na (14.26 g, 620 mmol) in EtOH (600.00 mL)). The mixture was stirred at 25° C. for 1 h. The reaction mixture was concentrated at low pressure. The residue was purified by silica gel column (PE/EA/TEA=100/1/1) to give the title compound (66.00 g, 300.99 mmol, 43.69% yield) as a yellow oil. ¹H NMR (400 MHz, CDCl₃) δ=4.27 (q, J=7.13 Hz, 2H), 3.56-3.67 (m, 2H), 3.48 (dq, J=9.65, 7.07 Hz, 2H), 2.35 (s, 6H), 1.33 (t, J=7.17 Hz, 3H), 1.23 (t, J=7.17 Hz, 6H).

### Intermediate 2. 3-((4-Methoxybenzyl)oxy)isothiazole

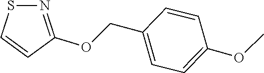

To a solution of isothiazol-3-ol (2.50 g, 24.72 mmol) in DMF (20.00 mL) was added K₂CO₃ (6.83 g, 49.44 mmol) under N2 at 0° C., and 4-methoxybenzyl chloride (PMB-CI) (4.26 g, 27.19 mmol, 3.70 mL, 1.10 eq.), and stirred at 25° C. for 18 h. The reaction mixture was quenched with H₂O (50 mL) and extracted with EtOAc (50×3 mL). The organic layer was washed with brine, dried over anhydrous Na₂SO₄, filtered and concentrated under reduced pressure. The residue was purified by silica gel column chromatography (FCC, SiO₂, PE/EA=100/1 to 40/1) to give the title compound (6.30 g, 25.91 mmol, 52.41% yield, 91% purity) as white solid. ¹H NMR (400 MHz, CDCl₃) δ=8.45 (d, J=4.9 Hz, 1H), 7.45-7.38 (m, 2H), 6.98-6.89 (m, 2H), 6.62 (d, J=4.6 Hz, 1H), 5.35 (s, 2H), 3.83 (s, 3H).

### Intermediate 3: (3R,4R,5R)-3,4-Bis(benzyloxy)-5-((benzyloxy)methyl)tetrahydrofuran-2-ol

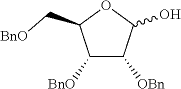 [SMILES: OC1O[C@H](COCc2ccccc2)[C@@H](OCc2ccccc2)[C@H]1OCc1ccccc1]

Step A: (2R,3S,4R)-2-(Hydroxymethyl)-5-methoxytetrahydrofuran-3,4-diol. To a solution of (3R,4S,5R)-5-(hydroxymethyl)tetrahydrofuran-2,3,4-triol (20.00 g, 133.22 mmol) in MeOH (150.00 mL) was added H₂SO₄ (2.40 g, 23.98 mmol, 1.30 mL, 98% purity). The reaction mixture was stirred at 25° C. for 12 hours. The reaction was set up for two batches. The reaction mixture was diluted with MeOH (200 mL), quenched with Na₂CO₃ solid and filtered. The filtrate was concentrated in vacuum. Purification (FCC, SiO₂, DCM/MeOH from 25/1 to 5/1) afforded the title compound (40 g, 243.67 mmol, 91.45% yield) as colorless oil.

Step B: (2R,3R,4R)-3,4-Bis(benzyloxy)-2-((benzyloxy)methyl)-5-methoxytetrahydrofuran. To a solution of 2R,3S,4R)-2-(hydroxymethyl)-5-methoxytetrahydrofuran-3,4-diol (20.00 g, 121.83 mmol) in DMF (200.00 mL) was added NaH (17.06 g, 426.41 mmol, 60% purity) at 0° C. The reaction mixture was stirred at 0° C. for 1 hour. Then TBAI (4.50 g, 12.18 mmol) was added and BnBr (72.93 g, 426.41 mmol, 50.65 mL, 3.50 eq.) was added dropwise into the solution. The reaction mixture was stirred at 25° C. for 11 hours. The reaction mixture was diluted with water (200 mL) and quenched with saturated NH₄Cl solution (200 mL). The resulting solution was extracted with EA (200 mL). The combined organic layers were washed with brine (200 mL×2), dried over anhydrous Na₂SO₄, filtered and concentrated under reduced pressure. The residue was purified by column chromatography (FCC, SiO₂, Petroleum ether/Ethyl acetate=25/1 to 5/1) to give the title compound (38.60 g, 88.83 mmol, 72.92% yield) as light yellow oil. ¹H NMR (400 MHz, CDCl₃) δ=7.38-7.23 (m, 14H), 4.91 (s, 1H), 4.68-4.63 (m, 1H), 4.62-4.58 (m, 1H), 4.55 (d, J=4.6 Hz, 1H), 4.53-4.51 (m, 1H), 4.46-4.41 (m, 1H), 4.36-4.30 (m, 1H), 4.00 (dd, J=4.6, 7.1 Hz, 1H), 3.83 (dd, J=0.7, 4.6 Hz, 1H), 3.63-3.56 (m, 1H), 3.52-3.46 (m, 1H), 3.30 (s, 3H).

Step C: (3R,4R,5R)-3,4-Bis(benzyloxy)-5-((benzyloxy)methyl)tetrahydrofuran-2-ol. (2R,3R,4R)-3,4-bis(benzyloxy)-2-((benzyloxy)methyl)-5-methoxytetrahydrofuran (25.00 g, 57.53 mmol) was dissolved in a mixture of TFA (70.00 mL) and H₂O (30.00 mL). The reaction mixture was stirred at 25° C. for 12 hours. The reaction was set up for three batches. The reaction mixture was diluted with water (300 mL) and neutralized with solid NaHCO₃ (120 g). The resulting solution was extracted with EA (500 mL). The organic layers were washed with brine (400 mL×2), dried over anhydrous Na₂SO₄, filtered and concentrated under reduced pressure. The residue was purified by column chromatography (FCC, SiO₂, Petroleum ether/Ethyl acetate=30:1 to 10:1) to give the title compound (56.50 g, 134.36 mmol, 77.85% yield) as colorless oil.

### Intermediate 4: (2S,3S,4R,5R)-3,4-Bis(benzyloxy)-5 ((benzyloxy)methyl)tetrahydrofuran-2-carbonitrile

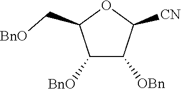 [SMILES: N#C[C@@H]1O[C@H](COCc2ccccc2)[C@@H](OCc2ccccc2)[C@H]1OCc1ccccc1]

Step A: (3R,4R,5R)-3,4-Bis(benzyloxy)-5-((benzyloxy)methyl)tetrahydrofuran-2-yl acetate. To a solution of (3R,4R,5R)-3,4-bis(benzyloxy)-5-((benzyloxy)methyl)tetrahydrofuran-2-ol (Intermediate 3, 35.00 g, 83.23 mmol) in DCM (500 mL) was added DMAP (1.02 g, 8.33 mmol) and Ac₂O (25.48 g, 249.69 mmol, 23.38 mL), and Et₃N (25.27 g, 107.01 mmol, 34.65 mL) at 0° C. The mixture was stirred at 25° C. of 1 h. The reaction mixture was quenched by addition of NaHCO₃ (50 mL), and diluted with EA (50 mL). The resulting solution was extracted with EA (200 mL×3). The combined organic layers were washed with brine (300 mL), dried over anhydrous Na₂SO₄, filtered and concentrated under reduce pressure. The residue was purified by column chromatography (FCC, SiO₂, PE/EA from 100/1 to 5/1) to give the title compound (32.00 g, 65.73 mmol, 78.97% yield, 95% purity) as colorless oil. LCMS: ESI-MS: m/z 485.2 [M+Na]⁺

Step B: (2S,3S,4R,5R)-3,4-Bis(benzyloxy)-5-((benzyloxy)methyl)tetrahydrofuran-2-carbonitrile. To a solution of (3R,4R,5R)-3,4-bis(benzyloxy)-5-((benzyloxy)methyl)tetrahydrofuran-2-yl acetate (30.00 g, 64.85 mmol) in CH₃CN (300.00 mL) was added trimethylsilyl cyanide (TMSCN) (9.65 g, 97.25 mmol), and BF₃·Et₂O (11.05 g, 77.80 mmol) at −35° C. The mixture was stirred at −35° C. for 1 h. The reaction was quenched with saturated NaHCO₃ solution (200 mL), and the reaction mixture was extracted with EA (200 mL×2). The organic layer was washed with brine (150 mL), dried over anhydrous Na₂SO₄, and concentrated at low pressure. The residue was purified by column (PE/EA from 20/1 to 4/1) to give the title compound (12.10 g, 27.89 mmol, 43.01% yield) as yellow oil. ¹H NMR (400 MHz, CDCl₃) δ 7.39-7.26 (m, 15H), 4.64-4.48 (m, 7H), 4.31 (t, J=5.0 Hz, 1H), 4.24 (q, J=3.7 Hz, 1H), 4.08-4.03 (m, 1H), 3.61-3.48 (m, 2H).

### Intermediate 5: Ethyl 2-((2R,3R,4R,5R)-3,4-bis(benzyloxy)-5-((benzyloxy)methyl)tetrahydrofuran-2-yl)-5-(trimethylstannyl)thiazole-4-carboxylate

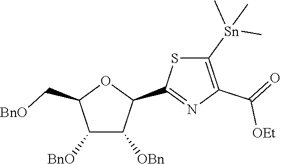 [SMILES: CCOC(=O)c1nc([C@@H]2O[C@H](COCc3ccccc3)[C@@H](OCc3ccccc3)[C@H]2OCc2ccccc2)s[c]1[Sn]([CH3])([CH3])[CH3]]

Step A: (2R,3R,4R,5R)-3,4-Bis(benzyloxy)-5-((benzyloxy)methyl)tetrahydrofuran-2-carbothioamide. A mixture of (2S,3S,4R,5R)-3,4-bis(benzyloxy)-5 ((benzyloxy)methyl)tetrahydrofuran-2-carbonitrile (Intermediate 4, 23.00 g, 53.55 mmol), Et₃N (55.00 mL) and EtOH (1.00 L) was bubbled with H₂S (15 PSI) at 18° C. for 2 h. The solvent was removed under reduced pressure. The residue was purified by column chromatography (FCC, SiC₂, Petroleum ether/Ethyl acetate=10/1) to give the title compound (24.00 g, 51.77 mmol, 96.68% yield, 100% purity) as a yellow solid. ¹H NMR (400 MHz, CDCl₃) δ 9.10 (br, s, 1H), 7.54-7.46 (m, 2H), 7.40-7.27 (m, 9H), 7.25-7.20 (m, 2H), 7.15 (dd, J=2.4, 7.1 Hz, 2H), 7.09 (br s, 1H), 4.97 (s, 1H), 4.91 (d, J=12.1 Hz, 1H), 4.71 (d, J=12.1 Hz, 1H), 4.51-4.45 (m, 2H), 4.41-4.36 (m, 1H), 4.34 (dd, J=1.6, 9.8 Hz, 1H), 4.30 (d, J=4.5 Hz, 1H), 4.17 (d, J=11.9 Hz, 1H), 3.99-3.92 (m, 2H), 3.63 (d, J=10.0 Hz, 1H). LCMS: ESI-MS: m/z=464.0 [M+H]⁺.

Step B: Ethyl 2-((2R,3R,4R,5R)-3,4-bis(benzyloxy)-5-((benzyloxy)methyl)tetrahydrofuran-2-yl)thiazole-4-carboxylate. A mixture of (2R,3R,4R,5R)-3,4-bis(benzyloxy)-5-((benzyloxy)methyl)tetrahydrofuran-2-carbothioamide (24.00 g, 51.77 mmol) and ethyl 3-bromo-2-oxo-propanoate (20.19 g, 103.54 mmol, 12.94 mL) in EtOH (300.00 mL) was refluxed for 5 h. The reaction mixture was concentrated under reduced pressure. The residue was purified by flash silica gel chromatography (ISCO®; 80 g Sepa Flash® Silica Flash Column, Eluent of 10% Ethyl acetate/Petroleum ether gradient @ 60 mL/min) to give the title compound (25.00 g, 34.84 mmol, 67.30% yield, 78% purity) as a colorless oil. ¹H NMR (400 MHz, CDCl₃) δ 8.09 (s, 1H), 7.42-7.26 (m, 15H), 5.46 (d, J=3.2 Hz, 1H), 4.85-4.70 (m, 2H), 4.65-4.50 (m, 3H), 4.49-4.35 (m, 4H), 4.30-4.25 (m, 1H), 3.95-3.90 (m, 1H), 3.77-3.65 (m, 2H), 1.43 (t, J=6.8 Hz, 3H).

Step C: Ethyl 2-((2R,3R,4R,5R)-3,4-bis(benzyloxy)-5-((benzyloxy)methyl)tetrahydrofuran-2-yl)-5-(trimethylstannyl)thiazole-4-carboxylate. LDA (2 M, 464.56 μL) was added to a solution of ethyl 2-((2R,3R,4R,5R)-3,4-bis(benzyloxy)-5-((benzyloxy)methyl)tetrahydrofuran-2-yl)thiazole-4-carboxylate (520.00 mg, 929.12 μmol) in anhydrous THF (5 mL) at −78° C. After 3 min., chlorotrimethylstannane (1 M, 2.32 mL) was added. The resulting mixture was stirred at −78° C. for another 15 min. The mixture was quenched with 10% citrate butter (pH=4.0, 5 mL) at −78° C. The reaction mixture was then brought to 18° C. and partitioned between 10% citrate buffer (pH=4.0, 5 mL) and EtOAc (15 mL). The organic layer was washed with brine, dried over anhydrous Na₂SO₄, filtered and concentrated. The residue was purified by column chromatography (FCC, SiO₂, Petroleum ether/Ethyl acetate=10/1) to give the title compound (3.40 g, 4.38 mmol, 42.82% yield, 93% purity) as an oil. LCMS: ESI-MS: m/z=723.8 [M+H]⁺. Note: Reactions (11 batches in parallel) and purified the combined residues once.

### Intermediate 6: Ethyl 2-((2R,3R,4R,5R)-3,4-bis(benzyloxy)-5-((benzyloxy)methyl)tetrahydrofuran-2-yl)-5-(tributylstannyl)thiazole-4-carboxylate

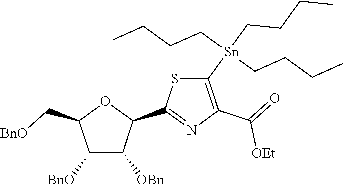 [SMILES: CCC[CH2][Sn]([CH2]CCC)([CH2]CCC)[c]1sc([C@@H]2O[C@H](COCc3ccccc3)[C@@H](OCc3ccccc3)[C@H]2OCc2ccccc2)nc1C(=O)OCC]

LDA (2 M, 984.50 μL) was added to a solution of ethyl 2-((2R,3R,4R,5R)-3,4-bis(benzyloxy)-5-((benzyloxy)methyl)tetrahydrofuran-2-yl)thiazole-4-carboxylate (Intermediate 5, product from Step B, 1.00 g, 1.79 mmol) in THF (10 mL) at −78° C. After 5 minutes, tributylchlorostannane (1.76 g, 5.41 mmol, 1.45 mL) was added. The resulting mixture was stirred at −78° C. for another 15 minutes. The mixture was quenched with 10% citrate buffer (pH=4.0, 10 mL) at −78° C. The reaction mixture was then brought to 18° C. and partitioned between 10% citrate buffer (pH=4.0, 10 mL) and EtOAc (50 mL). The organic layer was washed with brine, dried over anhydrous Na₂SO₄, filtered and concentrated. The residue was purified by column chromatography (FCC, SiO₂, Petroleum ether/Ethyl acetate=12/1) to give the title compound (6.40 g, 7.54 mmol, 42.13% yield) as an oil. LCMS: ESI-MS: m/z=850.4 [M+H]⁺, 872.3 [M+Na]⁺.

Reaction (10 batches in parallel) were combined and purified.

### Intermediate 7: (3R,4R,5R)-3,4-Bis(benzyloxy)-5-((benzyloxy)methyl)dihydrofuran-2 (3H)-one

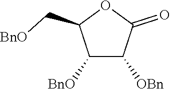 [SMILES: O=C1O[C@H](COCc2ccccc2)[C@@H](OCc2ccccc2)[C@H]1OCc1ccccc1]

To a solution of (3R,4R,5R)-3,4-bis(benzyloxy)-5-((benzyloxy)methyl)tetrahydrofuran-2-ol (Intermediate 3, 15.00 g, 35.67 mmol) in DCM (100.00 mL) was added pyridinium chlorochromate (PCC) (15.38 g, 71.34 mmol). The reaction was stirred at 40° C. for 12 hours. The reaction was set up for three batches. The reaction mixture was filtered on Celite®. The filtrate was concentrated in vacuum. The residue was purified by silica column chromatography (Petroleum ether/Ethyl acetate=30/1 to 5/1) to give the title compound (34.50 g, 82.44 mmol, 77.04% yield) as colorless oil. ¹H NMR (400 MHz, CDCl₃) δ=7.41-7.26 (m, 13H), 7.20-7.16 (m, 2H), 4.96 (d, J=11.9 Hz, 1H), 4.78-4.68 (m, 2H), 4.58-4.53 (m, 2H), 4.52-4.48 (m, 1H), 4.44-4.39 (m, 2H), 4.19-4.08 (m, 1H), 3.73-3.63 (m, 1H), 3.59-3.52 (m, 1H).

### Intermediate 8: 1-((2R,3R,4R,5R)-3,4-Bis(benzyloxy)-5-((benzyloxy)methyl)tetrahydrofuran-2-yl)-2-bromoethan-1-one

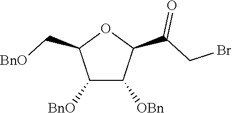 [SMILES: O=C(CBr)[C@@H]1O[C@H](COCc2ccccc2)[C@@H](OCc2ccccc2)[C@H]1OCc1ccccc1]

Step A: (2R,3R,4R,5R)-3,4-Bis(benzyloxy)-5-((benzyloxy)methyl)tetrahydrofuran-2-carboxylic acid. To a solution of (2S,3S,4R,5R)-3,4-bis(benzyloxy)-5 ((benzyloxy)methyl)tetrahydrofuran-2-carbonitrile (Intermediate 4, 10 g, 23.28 mmol) in H₂O (10 mL) and dioxane (60 mL) was added 4 M HCl in dioxane (80 mL) in one portion. The mixture was stirred at 80° C. for 12 h. The reaction mixture was concentrated at low pressure. The residue was dissolved in EA (100 mL), and the organic layer was washed with brine (50 mL), dried over anhydrous Na₂SO₄ and concentrated at low pressure. The residue was purified by column chromatography (FCC, SiO₂, Petroleum ether/Ethyl acetate=20/1 to 1:1) to give the title compound (6.8 g, 15.16 mmol, 65.12% yield) as yellow oil.

Step B: (2R,3R,4R,5R)-3,4-Bis(benzyloxy)-5-((benzyloxy)methyl)-N-methoxy-N-methyltetrahydrofuran-2-carboxamide. To a solution (2R,3R,4R,5R)-3,4-bis(benzyloxy)-5-((benzyloxy)methyl)tetrahydrofuran-2-carboxylic acid (6.5 g, 14.49 mmol) in THF (50 mL) was added DIPEA (11.24 g, 86.96 mmol, 15.19 mL) and HATU (6.61 g, 17.39 mmol), and N,O-dimethylhydroxylamine hydrochloride (4.24 g, 43.48 mmol). The mixture was stirred at 25° C. for 3 h. The reaction was quenched with H₂O (20 mL). The resulting solution was extracted by EA (20 mL×2) and the organic layer was washed with brine (20 mL) and dried over anhydrous Na₂SO₄, and concentrated at low pressure. The residue was purified by column chromatography (FCC, SiO₂, Petroleum ether/Ethyl acetate=20/1 to 3:1) to afford the title compound (6.2 g, 87.03% yield) was obtained as colorless oil. LCMS: ESI-MS: m/z=492.2 [M+H]⁺, 514.1 [M+Na]+

Step C: 1-((2R,3R,4R,5R)-3,4-Bis(benzyloxy)-5-((benzyloxy)methyl)tetrahydrofuran-2-yl)ethan-1-one. To a solution of (2R,3R,4R,5R)-3,4-bis(benzyloxy)-5-((benzyloxy)methyl)-N-methoxy-N-methyltetrahydrofuran-2-carboxamide (6 g, 12.21 mmol) in THF (100 mL) was added MeMgBr (3 M, 6.10 mL, 1.50 eq) at −78° C. The mixture was stirred at −78° C. for 1.5 h. The reaction was quenched with sat. NH₄Cl solution (30 mL), and the reaction mixture was extracted by EA (50 mL*2). The organic layer was washed with brine (35 mL), and dried over anhydrous Na₂SO₄, and concentrated at low pressure. The residue was purified by column chromatography (FCC, SiO₂, Petroleum ether/Ethyl acetate=50/1 to 3:1) to give the title compound (5.1 g, 93.57% yield) was obtained as yellow oil. ¹H NMR (400 MHz, CDCl₃) δ=7.38-7.14 (m, 15H), 4.66-4.38 (m, 7H), 4.28 (td, J=3.5, 6.6 Hz, 1H), 4.07-3.99 (m, 1H), 3.81 (dd, J=5.1, 6.4 Hz, 1H), 3.67 (dd, J=3.1, 10.6 Hz, 1H), 3.53 (dd, J=4.0, 10.6 Hz, 1H), 2.25-2.10 (m, 3H)

Step D: 1-((2R,3R,4R,5R)-3,4-Bis(benzyloxy)-5-((benzyloxy)methyl)tetrahydrofuran-2-yl)-2-bromoethan-1-one. To a solution 1-((2R,3R,4R,5R)-3,4-bis(benzyloxy)-5-((benzyloxy)methyl)tetrahydrofuran-2-yl)ethan-1-one (2 g, 4.48 mmol) in DCM (20 mL) was added DIPEA (2.32 g, 17.92 mmol, 3.13 mL) and followed by TMSOTf (2.99 g, 13.44 mmol, 2.43 mL) at 0° C. and stirred for 30 min. The reaction was quenched with water (20 mL), and diluted with DCM (20 mL). The organic phase was dried over anhydrous Na₂SO₄, concentrated at low pressure. The residue was dissolved in THF (10 mL) and H₂O (5 mL), followed by adding NBS (797.16 mg, 4.48 mmol, 1.00 eq) by portions at 0° C., and the mixture was stirred at 0° C. for 1 h. The reaction mixture was diluted with EA (30 mL) and water (20 mL). The organic layer was dried over anhydrous Na₂SO₄, and concentrated at low pressure. The residue was purified by column chromatography (FCC, SiO₂, Petroleum ether/Ethyl acetate=20/1 to 3:1) to give the title compound (3.3 g, 70.11% yield) d as yellow oil. ¹H NMR (400 MHz, CDCl₃) δ=7.35-6.99 (m, 15H), 4.70-4.19 (m, 7H), 4.19-4.10 (m, 3H), 4.10-4.00 (m, 1H), 3.74 (dd, J=5.0, 6.7 Hz, 1H), 3.59 (dd, J=2.6, 10.8 Hz, 1H), 3.47-3.27 (m, 1H); LCMS: ESI-MS: m/z=547.0, 549.0 [M+Na]⁺.

### Example 1: 5-((2R,3R,4S,5R)-3,4-Dihydroxy-5-(hydroxymethyl)tetrahydrofuran-2-yl)-1,2,4-thiadiazole-3-carboxamide

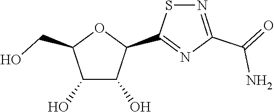 [SMILES: NC(=O)c1nsc([C@@H]2O[C@H](CO)[C@@H](O)[C@H]2O)n1]

Step A: (2R,3R,4R,5R)-3,4-Bis(benzyloxy)-5-((benzyloxy)methyl)tetrahydrofuran-2-carbothioamide. To a solution of (2S,3S,4R,5R)-3,4-bis(benzyloxy)-5-((benzyloxy)methyl)tetrahydrofuran-2-carbonitrile (Intermediate 4, 10.00 g, 23.28 mmol) in EtOH (300 mL) and Et₃N (50.00 mL) was bubbling with H₂S (15 PSI), and stirred at 25° C. for 1.5 h. The reaction mixture was concentrated at low pressure. The residue was purified by chromatograph (PE/EA=4:1) to give the title compound (9.60 g, 20.29 mmol, 87.17% yield, 98% purity) as yellow solid. ¹H NMR (400 MHz, CDCl₃) δ 9.12 (br, s, 1H), 7.51 (br, d, J=6.8 Hz, 2H), 7.40-7.08 (m, 14H), 4.97 (s, 1H), 4.91 (d, J=12.1 Hz, 1H), 4.72 (d, J=12.1 Hz, 1H), 4.51-4.45 (m, 2H), 4.41-4.28 (m, 3H), 4.16 (d, J=11.9 Hz, 1H), 4.00-3.93 (m, 2H), 3.64 (d, J=10.6 Hz, 1H). LCMS: ESI-MS: m/z 464.0 [M+H]⁺, 486.1 [M+Na]⁺.

Step B: Ethyl (Z)-2-(((2R,3R,4R,5R)-3,4-bis(benzyloxy)-5-((benzyloxy)methyl)tetrahydrofuran-2-carbonothioyl)imino)-2-(dimethylamino)acetate. To a solution of (2R,3R,4R,5R)-3,4-bis(benzyloxy)-5-((benzyloxy)methyl)tetrahydrofuran-2-carbothioamide (2.00 g×5, 4.32 mmol×3) in CH₃CN (1.50 mL×3) was added ethyl 2-(dimethylamino)-2,2-diethoxyacetate (Intermediate 1, 3.79 g×3, 17.28 mmol×3). The reaction mixture in sealed tube was stirred at 78° C. for 20 min under microwave irradiation. The reaction mixture was concentrated at low pressure. The residue was purified by chromatograph (PE/EA=1/1) to give the title compound (1.3 g, 16% yield) as yellow oil. 2.6 g of the recovered starting material was recycled. ¹H NMR (400 MHz, CDCl₃), δ=7.37-7.12 (m, 15H), 4.98 (d, J=1.5 Hz, 1H), 4.77 (d, J=12.0 Hz, 1H), 4.60 (s, 1H), 4.56 (s, 1H), 4.52-4.47 (m, 1H), 4.46 (d, J=7.8 Hz, 1H), 4.33 (td, J=4.2, 8.3 Hz, 1H), 4.23 (d, J=12.0 Hz, 1H), 4.18-4.10 (m, 2H), 4.07 (dd, J=1.6, 4.9 Hz, 1H), 3.87 (dd, J=4.9, 8.4 Hz, 1H), 3.67 (d, J=4.3 Hz, 2H), 2.94 (s, 3H), 2.67 (s, 3H), 1.18 (t, J=7.2 Hz, 3H). LCMS: ESI-MS: m/z 591.1 [M+Na]⁺.

Step C: Ethyl 5-((2R,3R,4R,5R)-3,4-bis(benzyloxy)-5-((benzyloxy)methyl)tetrahydrofuran-2-yl)-1,2,4-thiadiazole-3-carboxylate. To a solution of ethyl (Z)-2-(((2R,3R,4R,5R)-3,4-bis(benzyloxy)-5-((benzyloxy)methyl)tetrahydrofuran-2-carbonothioyl)imino)-2-(dimethylamino)acetate (1.3 g, 2.32 mmol) in EtOH (10.00 mL) and pyridine (367.0 mg, 4.64 mmol) was added amino oxysulfonic acid (262.4, 2.32 mmol) in MeOH (3.00 mL). The mixture was stirred at 55° C. for 12 h. The reaction was concentrated at low pressure. The residue was purified by chromatograph (PE/EA=5/1) to give the title compound (670 mg, 50.1% yield) as a yellow oil. ¹H NMR (400 MHz, CDCl₃) δ=7.42-7.24 (m, 15H), 5.52 (d, J=3.1 Hz, 1H), 4.85-4.72 (m, 2H), 4.61-4.49 (m, 5H), 4.44-4.38 (m, 2H), 4.23 (dd, J=3.2, 4.7 Hz, 1H), 3.98 (dd, J=4.9, 7.3 Hz, 1H), 3.78 (dd, J=2.3, 10.9 Hz, 1H), 3.60 (dd, J=3.5, 10.8 Hz, 1H), 1.53-1.44 (m, 3H).

LCMS: ESI-MS: m/z 583.1 [M+Na]⁺.

Step D: 5-((2R,3R,4R,5R)-3,4-Bis(benzyloxy)-5-((benzyloxy)methyl)tetrahydrofuran-2-yl)-1,2,4-thiadiazole-3-carboxamide. To a solution of ethyl 5-((2R,3R,4R,5R)-3,4-bis(benzyloxy)-5-((benzyloxy)methyl)tetrahydrofuran-2-yl)-1,2,4-thiadiazole-3-carboxylate (670.00 mg, 1.19 mmol) in EtOH (5.00 mL) was added NH₃-EtOH (10.00 mL). The mixture was stirred at 40° C. for 12 h. The reaction mixture was concentrated at low pressure. The residue was purified by silica gel column (PE/EA=2/1) to give the title compound (405 mg, 67.08% yield) as colorless oil. ¹H NMR (400 MHz, CDCl₃) δ=7.38-7.24 (m, 15H), 7.15 (br, s, 1H), 5.80 (br, s, 1H), 5.48 (d, J=3.5 Hz, 1H), 4.80-4.69 (m, 2H), 4.62-4.43 (m, 4H), 4.43-4.39 (m, 1H), 4.25-4.20 (m, 1H), 4.25-4.20 (m, 1H), 4.00 (dd, J=4.7, 6.7 Hz, 1H), 3.77 (dd, J=2.6, 10.8 Hz, 1H), 3.60 (dd, J=3.4, 10.9 Hz, 1H). LCMS: ESI-MS: m/z 532.1 [M+H]⁺ 554.1 [M+Na]⁺.

Step E: 5-((2R,3R,4S,5R)-3,4-Dihydroxy-5-(hydroxymethyl)tetrahydrofuran-2-yl)-1,2,4-thiadiazole-3-carboxamide. To a solution of 5-((2R,3R,4R,5R)-3,4-bis(benzyloxy)-5-((benzyloxy)methyl)tetrahydrofuran-2-yl)-1,2,4-thiadiazole-3-carboxamide (405.00 mg, 763 μmol) in DCM (2.00 mL) was added BCl₃ (1 M, 5.87 mL) at −78° C. The mixture was stirred at 0° C. for 2 h. The reaction was quenched with MeOH (5 mL), and stirred at 0° C. for 1 h, then concentrated at low pressure. The residue was dissolved in MeOH (1 mL) and three drops of NH₃ in MeOH (7.0 M, 5 mL) and stirred for another 1 h. The reaction mixture was concentrated at low pressure. The residue was purified by column (DCM/MeOH from 15/1 to 5/1) to give the title compound (122.1 mg) as white solid (combined with another batch for lyophilization). ¹H NMR (D₂O, 400 MHz) δ=5.38 (d, J=4.8 Hz, 1H), 4.36-4.39 (t, J=4.8 Hz, 1H), 4.73-4.84 (m, 1H), 4.17-4.24 (m, 1H), 3.89-3.90 (dd, J=3.2, 12.8 Hz, 1H), 3.75-3.80 (dd, J₁=5.2, 12.8 Hz, 1H). LCMS: ESI-MS: m/z 261.8 [M+1]⁺.

### Example 2: 5-((2R,3R,4S,5R)-3,4-Dihydroxy-5-(hydroxymethyl)tetrahydrofuran-2-yl)isothiazole-3-carboxamide

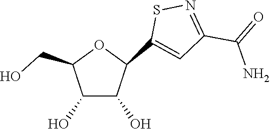 [SMILES: NC(=O)c1cc([C@@H]2O[C@H](CO)[C@@H](O)[C@H]2O)sn1]

Step A: (3R,4R,5R)-3,4-Bis(benzyloxy)-5-((benzyloxy)methyl)-2-(3-((4-methoxybenzyl)oxy)isothiazol-5-yl)tetrahydrofuran-2-ol. To a solution of LDA (2 M, 2.87 mL) in Et₂O (5.00 mL) was added dropwise 3-((4-methoxybenzyl)oxy)isothiazole (Intermediate 2, 1.27 g, 5.74 mmol) in THF (2.00 mL) at −78° C. under N2. After 30 minutes, (3R,4R,5R)-3,4-bis(benzyloxy)-5-((benzyloxy)methyl)dihydrofuran-2 (3H)-one (Intermediate 7, 2.00 g, 4.78 mmol) in THF (1.00 mL) was added, and the mixture was stirred at −78° C. for 2 h. The reaction mixture was quenched with saturated NH₄Cl solution (5 mL), and extracted with EtOAc (10×2 mL). The organic layer was washed with brine, dried with anhydrous Na₂SO₄, filtered and concentrated under reduced pressure. The residue was purified by column chromatography (FCC, SiO₂, Petroleum ether/Ethyl acetate=20/1 to 4/1) to give the title compound (1.04 g, 1.56 mmol, 32.65% yield, 96% purity) as yellow oil. LCMS: ESI-MS: m/z 662.0 [M+Na]⁺.

Step B: 5-((2R,3R,4R,5R)-3,4-Bis(benzyloxy)-5-((benzyloxy)methyl)tetrahydrofuran-2-yl)isothiazol-3-ol. To a solution of (3R,4R,5R)-3,4-bis(benzyloxy)-5-((benzyloxy)methyl)-2-(3-((4-methoxybenzyl)oxy)isothiazol-5-yl)tetrahydrofuran-2-ol (800.0 mg, 1.25 mmol) in DCM (20 mL) was added Et₃SiH (5.09 g, 43.75 mmol, 6.97 mL) and BF₃·Et₂O (887 mg, 6.25 mmol, 0.77 mL) at −78° C. under N2. The mixture was stirred at 25° C. for 5 h. The reaction mixture was quenched with H₂O (3 mL), and adjusted pH=7 with a solution of NaHCO₃ (10 mL). The resulting solution was extracted with DCM (20 mL×3), and the combined organic layers was dried over anhydrous Na₂SO₄, filtered and concentrated under reduced pressure. The residue was purified by column chromatography (FCC, SiO₂, Petroleum ether/Ethyl acetate=20/1 to 8/1) to give the title compound (372.0 mg, 664.8 μmol, 53.18% yield, 90% purity) as white solid. LCMS: ESI-MS: m/z 526.0 [M+Na]⁺.

Step C: 5-((2R,3R,4R,5R)-3,4-Bis(benzyloxy)-5-((benzyloxy)methyl)tetrahydrofuran-2-yl)isothiazol-3-yl trifluoromethanesulfonate. To a solution of 5-((2R,3R,4R,5R)-3,4-bis(benzyloxy)-5-((benzyloxy)methyl)tetrahydrofuran-2-yl)isothiazol-3-ol (372.0 mg, 738.7 μmol) in DCM (5 mL) was added pyridine (py) (350.6 mg, 4.4 mmol, 357.7 μL, 6.00 eq.) and Tf₂O (312.6 mg, 1.1 mmol, 182.8 μL, 1.50 eq.) dropwise at −30° C. under N2. The mixture was stirred at −30° C. for 1 h. The reaction mixture was washed with a solution of NaHCO₃ (2 mL), then extracted with DCM (2 mL×3). The combined organic layers were dried over anhydrous Na₂SO₄, filtered and concentrated under reduced pressure. The residue was purified by column chromatography (FCC, SiO₂, Petroleum ether/Ethyl acetate=20/1 to 12/1) to give the title compound (300.0 mg, 471.9 μmol, 63.89% yield) as colorless oil. ¹H NMR (400 MHz, CDCl₃) δ=7.25 (br, s, 15H), 6.83 (s, 1H), 5.25 (d, J=7.5 Hz, 1H), 4.65-4.34 (m, 7H), 4.04-3.98 (m, 1H), 3.86 (dd, J=5.0, 7.4 Hz, 1H), 3.61-3.50 (m, 2H). LCMS: ESI-MS: m/z 635.9 [M+1]⁺.

Step D: 5-((2R,3R,4R,5R)-3,4-Bis(benzyloxy)-5-((benzyloxy)methyl)tetrahydrofuran-2-yl)isothiazole-3-carbonitrile. To a solution of 5-((2R,3R,4R,5R)-3,4-bis(benzyloxy)-5-((benzyloxy)methyl)tetrahydrofuran-2-yl)isothiazol-3-yl trifluoromethanesulfonate (230.0 mg, 361.8 μmol) in DMF (1.50 mL) was added Zn(CN)₂ (85 mg, 723.6 μmol, 45.9 μL), Pd₂(dba)₃ (132.5 mg, 144.7 μmol), DPPF (120.3 mg, 217.1 μmol). The mixture was stirred at 65° C. for 5 h. The reaction mixture was filtered, and the filtrate was washed with H₂Oand saturated brines (10 mL, 1:1), then extracted with EA (10 mL×3). The combined organic layers were dried over anhydrous Na₂SO₄, filtered and concentrated under reduced pressure. The residue was purified by column chromatography (FCC, SiO₂, Petroleum ether/Ethyl acetate=20/1 to 12/1) to give the title compound (160.00 mg, 277.79 μmol, 76.78% yield, 89% purity) as yellow oil. LCMS: ESI-MS: m/z 513.2 [M+1]⁺, m/z 535.2 [M+Na]⁺.

Step E: 5-((2R,3R,4R,5R)-3,4-Bis(benzyloxy)-5-((benzyloxy)methyl)tetrahydrofuran-2-yl)isothiazole-3-carboxamide. 5-((2R,3R,4R,5R)-3,4-bis(benzyloxy)-5-((benzyloxy)methyl)tetrahydrofuran-2-yl)isothiazole-3-carbonitrile (160.0 mg, 312.1 mol) was dissolved in a mixture of MeOH (400.00 μL), NH₃—H₂O (7.10 g, 56.7 mmol, 7.8 mL, 28% purity) and H₂O₂ (933.0 mg, 8.2 mmol, 790.7 μL, 30% purity) in one portion at 25° C. under N2. The mixture was stirred at 25° C. for 4 h. The reaction mixture was quenched with saturated Na₂SO₃ solution (6 mL). The resulting solution was extracted with EtOAc (10×3 mL). The organic layer was washed with brine, dried with anhydrous Na₂SO₄, filtered and concentrated under reduced pressure. The residue was purified by column chromatography (FCC, SiO₂, PE: EA=20:1 to 3:1) to give the title compound (130.0 mg, 73.39% yield, 93.5% purity) as yellow solid. ¹H NMR (400 MHz, CDCl₃) δ=7.71 (s, 1H), 7.41-7.23 (m, 15H), 7.09 (br, s, 1H), 5.53 (br, s, 1H), 5.33 (d, J=6.6 Hz, 1H), 4.62-4.47 (m, 6H), 4.37 (br, d, J=3.3 Hz, 1H), 3.99 (t, J=4.0 Hz, 1H), 3.92-3.84 (m, 1H), 3.64-3.51 (m, 2H). LCMS: ESI-MS: m/z 553.0 [M+Na]⁺.

Step F: 5-((2R,3R,4S,5R)-3,4-Dihydroxy-5-(hydroxymethyl)tetrahydrofuran-2-yl)isothiazole-3-carboxamide. To a solution of 5-((2R,3R,4R,5R)-3,4-bis(benzyloxy)-5-((benzyloxy)methyl)tetrahydrofuran-2-yl)isothiazole-3-carboxamide (110.0 mg, 207.3 mol) in DCM (2.00 mL) was added BCl₃ (1 M, 2.07 mL) at −78° C. under N2. The mixture was stirred at 0° C. for 2 h. The reaction mixture was quenched with MeOH (2 mL) and NH₃·H₂O (0.5 mL). The reaction mixture was stirring for 1 h, and concentrated in vacuum. The residue was purified by column chromatography (FCC, SiO₂, DCM/MeOH=30/1 to 10/1) to give the title compound (47.0 mg, 178.8 μmol, 86.24% yield, 99% purity) as light yellow solid. ¹H NMR (400 MHz, CD₃OD) δ=7.74 (d, J=0.9 Hz, 1H), 5.10 (d, J=7.1 Hz, 1H), 4.11-4.00 (m, 2H), 3.93 (dd, J=5.3, 6.8 Hz, 1H), 3.77-3.65 (m, 2H). MS: ESI-MS: m/z 261.05 [M+H]⁺.

### Example 3: 2-((2R,3R,4S,5R)-3,4-Dihydroxy-5-(hydroxymethyl)tetrahydrofuran-2-yl)-5-fluorothiazole-4-carboxamide

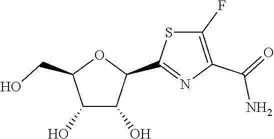 [SMILES: NC(=O)c1nc([C@@H]2O[C@H](CO)[C@@H](O)[C@H]2O)sc1F]

Step A:

Method A: Ethyl 2-((2R,3R,4R,5R)-3,4-bis(benzyloxy)-5-((benzyloxy)methyl)tetrahydrofuran-2-yl)-5-fluorothiazole-4-carboxylate. A stirred mixture of ethyl 2-((2R,3R,4R,5R)-3,4-bis(benzyloxy)-5-((benzyloxy)methyl)tetrahydrofuran-2-yl)-5-(trimethylstannyl)thiazole-4-carboxylate (Intermediate 5, 800.00 mg, 1.11 mmol), NaHCO₃ (186.05 mg, 2.21 mmol, 86.13 μL), Ag₂O (25.66 mg, 110.73 μmol), AgOTf (341.41 mg, 1.33 mmol) and Select-Fluor (786.46 mg, 2.22 mmol, 2.00 eq.) in acetone (60.00 mL) in a sealed vessel was heated at 65° C. for 4 h and shielded from light. The reaction mixture was filtered through a short pad of Celite® and washed with acetone (50 mL). The filtrate was concentrated under reduced pressure. The residue was diluted with EA (50 mL) and washed with sat·NaHCO₃ (45 mL), dried over Na₂SO₄, filtered and concentrated under reduced pressure. The residue was purified by column chromatography (FCC, SiO₂, Petroleum ether/Ethyl acetate=10/1) to give crude product the title compound (300.00 mg) as an oil. Three batches in parallel were set up and combined and purified.

Method B: Ethyl 2-((2R,3R,4R,5R)-3,4-bis(benzyloxy)-5-((benzyloxy)methyl)tetrahydrofuran-2-yl)-5-fluorothiazole-4-carboxylate. A stirred mixture of ethyl 2-((2R,3R,4R,5R)-3,4-bis(benzyloxy)-5-((benzyloxy)methyl)tetrahydrofuran-2-yl)-5-(tributylstannyl)thiazole-4-carboxylate (Intermediate 6, 1.00 g, 1.18 mmol), NaHCO₃ (198.26 mg, 2.36 mmol, 91.79 μL), Ag₂O (27.35 mg, 118.00 μmol), AgOTf (303.19 mg, 1.18 mmol) and Select-Fluor® (836.05 mg, 2.36 mmol) in acetone (60.00 mL) in a sealed vessel was heated at 65° C. for 4 h and shielded from light. The reaction mixture was filtered through a short pad of Celite® and washed with acetone (50 mL) and the filtrate was concentrated under reduced pressure. The residue was diluted with EA (50 mL) and washed with sat. aq. NaHCO₃ (45 mL). The resulting solution was dried over Na₂SO₄, filtered and concentrated under reduced pressure. The residue was purified by column chromatography (FCC, SiO₂, Petroleum ether/Ethyl acetate=10/1) to give crude product title compound (400 mg) as an oil. The products from Method A and Method B (700 mg) were combined and purified by Prep-HPLC (FA system) to give the title compound (450.00 mg, 777.45 μmol, 21.96% yield, 99.8% purity) as a colorless oil. ¹H NMR (400 MHz, CD₃OD) δ 7.43-7.26 (m, 15H), 5.26 (t, J=2.5 Hz, 1H), 4.83-4.69 (m, 2H), 4.66-4.49 (m, 3H), 4.48-4.39 (m, 1H), 4.49-4.39 (m, 2H), 4.36 (td, J=3.5, 6.7 Hz, 1H), 4.25 (dd, J=3.5, 4.8 Hz, 1H), 3.98 (dd, J=4.9, 6.9 Hz, 1H), 3.75 (dd, J=2.5, 10.8 Hz, 1H), 3.59 (dd, J=4.0, 10.8 Hz, 1H), 1.42 (t, J=7.2 Hz, 3H). ¹⁹F-NMR (376 MHz, CD₃OD), δ=−128.64. LCMS: ESI-MS: m/z=578.0 [M+H]⁺, 600.0 [M+Na]+

Step B: Ethyl 2-((2R,3R,4S,5R)-3,4-dihydroxy-5-(hydroxymethyl)tetrahydrofuran-2-yl)-5-fluorothiazole-4-carboxylate. To a solution of ethyl 2-((2R,3R,4R,5R)-3,4-bis(benzyloxy)-5-((benzyloxy)methyl)tetrahydrofuran-2-yl)-5-fluorothiazole-4-carboxylate (400.00 mg, 692.45 μmol) in DCM (7.00 mL) was added a solution of BCl₃ (1 M, 6.92 mL) at −78° C. The mixture was stirred at 0° C. for 30 minutes. The reaction mixture was quenched by addition of EtOH (1.5 mL) at −78° C., and then neutralized with NH₃—H₂O (1 mL). The mixture was concentrated under reduced pressure. The residue was purified by flash silica gel chromatography (ISCO®; 4 g Sepa Flash® Silica Flash Column, Eluent of 0-5% MeOH/DCM ether gradient @ 18 mL/min) to give the title compound (140.00 mg, 65.14% yield, 99% purity) as a white solid. ¹H NMR (400 MHz, CD₃OD) S=4.90 (dd, J=2.2, 4.9 Hz, 1H), 4.37 (q, J=7.1 Hz, 2H), 4.19 (t, J=4.7 Hz, 1H), 4.02 (m, 2H), 3.81-3.75 (m, 1H), 3.72-3.61 (m, 1H), 1.36 (t, J=7.1 Hz, 3H). LCMS: ESI-MS: m/z=307.8 [M+H]⁺.

Step C: 2-((2R,3R,4S,5R)-3,4-Dihydroxy-5-(hydroxymethyl)tetrahydrofuran-2-yl)-5-fluorothiazole-4-carboxamide. Ethyl 2-((2R,3R,4S,5R)-3,4-dihydroxy-5-(hydroxymethyl)tetrahydrofuran-2-yl)-5-fluorothiazole-4-carboxylate (115.00 mg, 374.24 mol) was treated with NH₃—H₂O (2.50 mL, 25-28%). The reaction mixture was stirred at 18° C. for 15 minutes. The reaction mixture was concentrated under reduced pressure. The residue was purified by Prep-HPLC (FA system) to give the title compound (51.60 mg, 49.55% yield, 100% purity) as a white solid. ¹H NMR (400 M Hz, D₂O) δ 5.01 (dd, J=1.8, 5.3 Hz, 1H), 4.35 (t, J=5.0 Hz, 1H), 4.20-4.16 (m, 1H), 4.16-4.12 (m, 1H), 3.88-3.82 (m, 1H), 3.77-3.70 (m, 1H). ¹⁹F-NMR (376 MHz, D₂O), δ=−132.44. LCMS: ESI-MS: m/z=278.9 [M+H]⁺.

### Example 4: ((2R,3S,4R,5R)-5-(3-Carbamoyl-1,2,4-thiadiazol-5-yl)-3,4-dihydroxytetrahydrofuran-2-yl)methyl isobutyrate

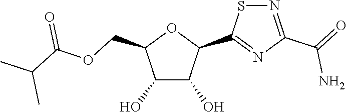 [SMILES: CC(C)C(=O)OC[C@H]1O[C@@H](c2nc(C(N)=O)ns2)[C@H](O)[C@@H]1O]

Step A: 5-((3aR,4R,6R,6aR)-6-(Hydroxymethyl)-2-methoxytetrahydrofuro[3,4-d][1,3]dioxol-4-yl)-1,2,4-thiadiazole-3-carboxamide. To a solution of 5-((2R,3R,4S,5R)-3,4-dihydroxy-5-(hydroxymethyl)tetrahydrofuran-2-yl)-1,2,4-thiadiazole-3-carboxamide (Example 2,120 mg, 459.33 μmol) in dioxane (2.00 mL) and DMF (400 μL) was added trimethoxymethane (389 mg, 3.67 mmol) and p-toluenesulfonic acid (TsOH) (31.6 mg, 183.73 μmol). The reaction mixture was stirred at 25° C. for 12 h. The reaction mixture was quenched by addition of Et₃N (1 mL), and concentrated under reduced pressure. The residue was purified by column chromatography (FCC, SiO₂, DCM/MeOH=20/1) to give the title compound (105 mg, 75.37% yield) as colorless oil.

Step B: ((3aR,4R,6R,6aR)-6-(3-Carbamoyl-1,2,4-thiadiazol-5-yl)-2-methoxytetrahydrofuro[3,4-d][1,3]dioxol-4-yl)methyl isobutyrate. To a solution of 5-((3aR,4R,6R,6aR)-6-(hydroxymethyl)-2-methoxytetrahydrofuro[3,4-d][1,3]dioxol-4-yl)-1,2,4-thiadiazole-3-carboxamide (105.00 mg, 346.20 μmol) in pyridine (1.00 mL) was added a solution of isobutyryl chloride (40.58 mg, 380.82 μmol) in DCM (1.00 mL) at 0° C. The mixture was stirred at 25° C. for 2 hr. The reaction was quenched by addition of MeOH (1 mL), and concentrated under reduced pressure. The residue was purified by column chromatography (FCC, SiO₂, EA/PE=1/0) to give the title compound (85.6 mg, 62.9% yield, 95% purity) as colorless oil. ESI-MS: m/z 395.9 [M+Na]⁺.

Step C: ((2R,3S,4R,5R)-5-(3-Carbamoyl-1,2,4-thiadiazol-5-yl)-3,4-dihydroxytetrahydrofuran-2-yl)methyl isobutyrate. ((3aR,4R,6R,6aR)-6-(3-Carbamoyl-1,2,4-thiadiazol-5-yl)-2-methoxytetrahydrofuro[3,4-d][1,3]dioxol-4-yl)methyl isobutyrate (86.00 mg, 230.33 μmol) was dissolved in dioxane (1.00 mL), and HCl/dioxane (1 M, 1.15 mL) and H₂O (8.30 mg, 460.66 μmol) was added. The mixture was stirred at 25° C. for 5 hr. The reaction mixture was quenched by addition of saturated NaHCO₃ (4 mL). The reaction mixture was concentrated under reduced pressure. The residue was purified by column chromatography (FCC, SiO₂, DCM/MeOH=30/1) to give a crude which was purified by Prep-HPLC (Phenomenex Gemini C18 250*50 10 μm; mobile phase: [water (0.225% FA)-ACN]; B %: 13%-43%,11.2 min) to afford the title compound (28.20 mg, 36.95% yield) as white solid. ¹H-NMR (400 MHz, CDCl₃), δ=7.52 (br, s, 1H), 6.81 (br, s, 1H), 5.32 (br, d, J=4.4 Hz, 1H), 4.42-4.36 (m, 2H), 4.35 (br, s, 1H),4.30-4.24 (m, 1H), 4.19 (br, s, 1H), 2.54 (td, J=7.0, 13.9 Hz, 1H), 1.13 (d, J=7.1 Hz, 6H). LCMS: ESI-MS: m/z 331.9 [M+H]⁺.

### Example 5

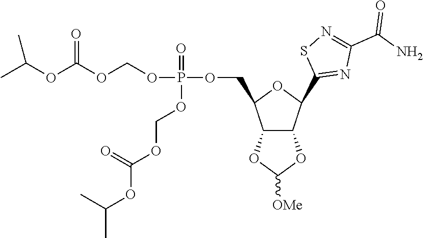

Step A. To a solution of 5-((2R,3R,4S,5R)-3,4-dihydroxy-5-(hydroxymethyl)tetrahydrofuran-2-yl)-1,2,4-thiadiazole-3-carboxamide (Example 1, 30 mg, 0.11 mmol) in dioxane (1 mL) were added trimethyl orthoformate (0.36 mL, 3.3 mmol) and p-toluenesulfonic acid monohydrate (21 mg, 0.11 mmol) and the resulting mixture stirred overnight at r. t. The mixture was then neutralized with methanolic ammonia, concentrated and purified by flash chromatography on silica with MeOH/CH₂Cl₂ solvent system (2-10% gradient) to yield 20 mg of 2′,3′-methoxymethylene derivative.

Step B. The intermediate from Step A is reacted with triethylammonium bis(POC)phosphate (0.14 mmol), DIPEA (61 μL), BopCl (54 mg) and nitrotriazole (24 mg) in THF (1 mL) into provide Example 5 (27 mg, 40% for 2 steps) in the same manner as described for Example 4. ³¹P-NMR (CDCl₃): δ−4.41 , −4.33. MS: m/z=616 (M+1)+.

### Example 6

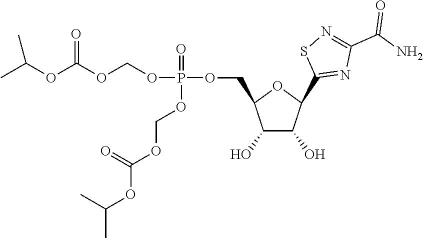 [SMILES: CC(C)OC(=O)OCOP(=O)(OCOC(=O)OC(C)C)OC[C@H]1O[C@@H](c2nc(C(N)=O)ns2)[C@H](O)[C@@H]1O]

A solution of Example 5 (27 mg, 0.044 mmol) and 80% aq. AcOH (2 mL) was stirred at r. t. for 3 h. The mixture was then concentrated. Thus obtained residue was co-evaporated several times with toluene followed by methanol containing few drops of Et₃N. The evaporated residue was purified on silica gel column with MeOH/CH₂Cl₂ solvent system (3-12% gradient) to yield Example 6 (20 mg, 80%). ¹H-NMR (CD₃CN): δ 7.39 (br s, 1H), 6.43 (br s, 1H), 5.62 (d, J=2.8 Hz, 1H), 5.59 (d, J=2.8 Hz, 1H), 5.23 (d, J=4.4 Hz, 1H) 4.88 (m, 2H), 4.34 (m, 1H), 4.17-4.26 (m, 3H), 4.08 (m, 2H), 4.08 (m, 2H), 3.74 (br s, 1H), 1.27 (m, 12H). ³¹P-NMR (CD₃CN): δ−4.38. MS: m/z=574 (M+1)+.

### Example 7: ((2R,3S,4R,5R)-5-(3-Carbamoyl-1,2,4-thiadiazol-5-yl)-3,4-dihydroxytetrahydrofuran-2-yl)methyl valinate

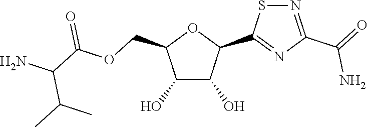 [SMILES: CC(C)C(N)C(=O)OC[C@H]1O[C@@H](c2nc(C(N)=O)ns2)[C@H](O)[C@@H]1O]

Step A: 5-((3aR,4R,6R,6aR)-6-(Hydroxymethyl)-2-methoxytetrahydrofuro[3,4-d][1,3]dioxol-4-yl)-1,2,4-thiadiazole-3-carboxamide. 5-((2R,3R,4S,5R)-3,4-Dihydroxy-5-(hydroxymethyl)tetrahydrofuran-2-yl)-1,2,4-thiadiazole-3-carboxamide (Example 1, 52 mg, 0.2 mmol) was dissolved in dioxane (2 mL). Methyl orthoformate was added (210 L, 2 mmol) followed by TsOH (76 mg, 0.4 mmol). The mixture was left overnight at ambient temperature. Methanol (5 mL) and Et₃N (0.5 mL) was added and left for 30 min. at ambient temperature. The reaction mixture was concentrated under reduced pressure. Purification (FCC, SiO₂, methanol in DCM from 2% to 10%) afforded 40 mg of the title compound.

Step B. ((3aR,4R,6R,6aR)-6-(3-Carbamoyl-1,2,4-thiadiazol-5-yl)-2-methoxytetrahydrofuro[3,4-d][1,3]dioxol-4-yl)methyl valinate. To a solution of 5-((3aR,4R,6R,6aR)-6-(hydroxymethyl)-2-methoxytetrahydrofuro[3,4-d][1,3]dioxol-4-yl)-1,2,4-thiadiazole-3-carboxamide (40 mg, 0.13 mmol)) in DMF (5 mL), was added Boc-valine (0.5 mmol) and DCC (0.5 mmol), and stirred for 20 hours. The reaction mixture was concentrated under reduced pressure. 5 mL of water was added. Urea was filtered out, and the filtrate was extracted with EA (10×3). The organic fraction was concentrated at low pressure. The residue was purified by flash chromatography on silica gel with MeOH/CH₂Cl₂ solvent system (2-10% gradient) to yield 2′,3′-methoxymethylene derivative.

Step C. ((2R,3S,4R,5R)-5-(3-Carbamoyl-1,2,4-thiadiazol-5-yl)-3,4-dihydroxytetrahydrofuran-2-yl)methyl valinate. A solution of ((3aR,4R,6R,6aR)-6-(3-carbamoyl-1,2,4-thiadiazol-5-yl)-2-methoxytetrahydrofuro[3,4-d][1,3]dioxol-4-yl)methyl valinate was treated with 1 N HCl/dioxane-DCM 1:1 (v/v) solution for 40 min., and concentrated at low pressure. The residue was purified by RP HPLC in 0.05 M formic acid to give the title compound (20 mg, 42%). ¹H-NMR (CD₃OD), δ=8.48 (br s, 1H), 6.43 (br s, 1H), 5.27 (d, 1H), 4.86-4.41 (m, 2H), 4.31-4.27 (m, 2H), 4.05-4.03 (dd, 1H), 3.93-3.91 (m, 1H), 2.20-2.30 (m, 1H), 1.05 (d, 6H). MS: m/z=362 (M+1)+.

### Example 8: Synthesis of Nucleoside 5′-triphosphates

Dry nucleoside (0.05 mmol) was dissolved in dry PO(OMe)₃ (0.7 mL) N-methylimidazole (0.009 mL, 0.11 mmol) was added followed by POCl₃ (0.009 mL, 0.11 mmol). The reaction mixture was stirred at rt for 20-40 minutes. The reaction was controlled by LCMS and monitored by the appearance of corresponding nucleoside 5′-monophosphate. After completion of the reaction, tetrabutylammonium salt of pyrophosphate (150 mg) was added, followed by DMF (0.5 mL) to get a homogeneous solution. After 1.5 hours at ambient temperature, the reaction was diluted with water (10 mL) and loaded on the column HiLoad 16/10 with Q Sepharose High Performance. Separation was done in a linear gradient of NaCl from 0 to 1 N in 50 mM TRIS-buffer (pH7.5). Triphosphate was eluted at 75-80% B. Corresponding fractions were concentrated. Desalting was achieved by RP HPLC on Synergy 4 micron Hydro-RP column (Phenominex). A linear gradient of methanol from 0 to 30% in 50 mM triethylammonium acetate buffer (pH 7.5) was used for elution. The corresponding fractions were combined, concentrated and lyophilized 3 times to remove excess of buffer.

### Example 9: ((2R,3S,4R,5R)-5-(4-Carbamoyl-5-fluorothiazol-2-yl)-3,4-dihydroxytetrahydrofuran-2-yl)methyl tetrahydrogen triphosphate

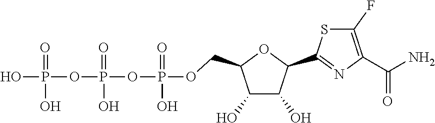 [SMILES: NC(=O)c1nc([C@@H]2O[C@H](COP(=O)(O)OP(=O)(O)OP(=O)(O)O)[C@@H](O)[C@H]2O)sc1F]

The title compound was prepared in a manner analogous to Example 8, using nucleoside described in Example 3. MS: m/z=516.7 (M−1)⁻. ³¹P-NMR (D₂O), δ=−11.05 (d), −11.65 (d), −23.47 (t).

### Example 10: ((2R,3S,4R,5R)-5-(3-Carbamoyl-1,2,4-thiadiazol-5-yl)-3,4-dihydroxytetrahydrofuran-2-yl)methyl tetrahydrogen triphosphate

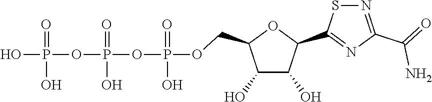 [SMILES: NC(=O)c1nsc([C@@H]2O[C@H](COP(=O)(O)OP(=O)(O)OP(=O)(O)O)[C@@H](O)[C@H]2O)n1]

The title compound was prepared in a manner analogous to Example 8, using nucleoside described in Example 1. MS: m/z=500.0 (M−1)⁻. ³¹P-NMR (D₂O), δ=−10.95 (d), −11.67 (d), −23.46 (t).

### Example 11: ((2R,3S,4R,5R)-5-(3-Carbamoylisothiazol-5-yl)-3,4-dihydroxytetrahydrofuran-2-yl)methyl tetrahydrogen triphosphate

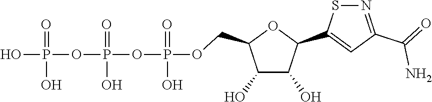 [SMILES: NC(=O)c1cc([C@@H]2O[C@H](COP(=O)(O)OP(=O)(O)OP(=O)(O)O)[C@@H](O)[C@H]2O)sn1]

The title compound was prepared in a manner analogous to Example 8, using nucleoside described in Example 2. MS: m/z=499.2 (M−1)⁻. ³¹P-NMR (D₂O), δ=−10.93 (d), −11.58 (d), −27.63 (t).

### Example 12: ((2R,3S,4R,5R)-5-(3-Carbamoyl-4-fluoroisothiazol-5-yl)-3,4-dihydroxytetrahydrofuran-2-yl)methyl tetrahydrogen triphosphate

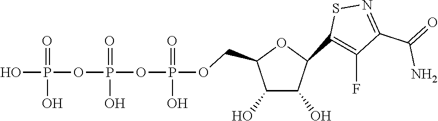 [SMILES: NC(=O)c1nsc([C@@H]2O[C@H](COP(=O)(O)OP(=O)(O)OP(=O)(O)O)[C@@H](O)[C@H]2O)c1F]

Step A: 5-((2R,3R,4R,5R)-3,4-Bis(benzyloxy)-5-((benzyloxy)methyl)tetrahydrofuran-2-yl)isothiazole-3-carbonitrile. The title compound is Example 3, product from Step D.

Step B: 5-((2R,3R,4R,5R)-3,4-Bis(benzyloxy)-5-((benzyloxy)methyl)tetrahydrofuran-2-yl)isothiazole-3-carboxylic acid. 5-((2R,3R,4R,5R)-3,4-Bis(benzyloxy)-5-((benzyloxy)methyl)tetrahydrofuran-2-yl)isothiazole-3-carbonitrile (2.4 g, 4.68 mmol, 1.00 eq.) was dissolved in a mixture of MeOH (20 mL), H₂O (2 mL) and THF (2 mL), and then KOH (1.05 g, 18.73 mmol, 4.00 eq) was added. The reaction mixture was stirred at 90° C. for 18 h. The reaction mixture was extracted with EA (20 mL×3), and the combined organic layers was dried over anhydrous Na₂SO₄, filtered and concentrated under reduced pressure. The residue was purified by column chromatography (SiO₂, DCM/MeOH=50/1 to 30/1) to give the title compound (3 g, 4.06 mmol, 86.78% yield, 90% purity) as yellow oil. LCMS: ESI-MS: m/z 532.3 [M+1]⁺, m/z 554.2 [M+Na]⁺.

Step C: tert-Butyl 5-((2R,3R,4R,5R)-3,4-bis(benzyloxy)-5-((benzyloxy)methyl)tetrahydrofuran-2-yl)isothiazole-3-carboxylate. To a solution of 5-((2R,3R,4R,5R)-3,4-bis(benzyloxy)-5-((benzyloxy)methyl)tetrahydrofuran-2-yl)isothiazole-3-carboxylic acid (1.29 g, 2.43 mmol, 1.00 eq) in DCM (10 mL) was added 2-methylpropan-2-ol (215.83 mg, 2.91 mmol, 278.49 μL, 1.2 eq) and DMAP (59.29 mg, 485.31 μmol, 0.20 eq), and DCC (751.01 mg, 3.64 mmol, 736.28 μL, 1.50 eq), and stirred at 25° C. for 2 h. The reaction was set up for two batches. The reaction mixture was filtered and the filter was concentrated under reduced pressure. The residue was purified by column chromatography (SiO₂, PE: EA=20:1 to 11:1) to give the title compound (1.4 g, 2.24 mmol, 46.14% yield, 94% purity) as light yellow oil. ¹H-NMR (400 MHz, CDCl₃), δ=7.61 (d, J=1.8 Hz, 1H), 7.39-7.23 (m, 15H), 5.34-5.29 (m, 1H), 4.61-4.55 (m, 4H), 4.54-4.45 (m, 2H), 4.37 (br s, 1H), 4.03-3.97 (m, 1H), 3.89-3.82 (m, 1H), 3.60-3.54 (m, 2H), 1.64 (d, J=1.8 Hz, 9H). LCMS:ESI-MS: m/z 610.0 [M+Na]⁺.

Step D: tert-Butyl 5-((2R,3R,4R,5R)-3,4-bis(benzyloxy)-5-((benzyloxy)methyl)tetrahydrofuran-2-yl)-4-(trimethylstannyl)isothiazole-3-carboxylate. To a solution of tert-butyl 5-((2R,3R,4R,5R)-3,4-bis(benzyloxy)-5-((benzyloxy)methyl)tetrahydrofuran-2-yl)isothiazole-3-carboxylate (490 mg, 833.72 mol) in THF (3.5 mL) was added LDA (2 M, 500.23 μL) at −78° C. under N2. The mixture was stirred for 5 minutes, chlorotrimethylstannane (415.33 mg, 2.08 mmol, 420.38 μL) was added dropwise. The mixture was stirred at −78° C. for 1 h. TLC (PE/EA=3/1) showed that the reaction was complete. The reaction mixture was treated with saturated KF solution (2 mL), and stirred for 0.5 h, and then adjusted pH=4 with critic acid. The resulting mixture was extracted with EA (20 mL×3). The combined organic layers was dried over anhydrous Na₂SO₄, filtered and concentrated under reduced pressure. The residue was purified by column chromatography (SiO₂, PE/EA=30/1 to 11/1) to give the title compound (320 mg, 46.54% yield, 91% purity) as colorless oil. LCMS: ESI-MS: m/z 774.0 [M+Na]⁺.

Step E: tert-Butyl 5-((2R,3R,4R,5R)-3,4-bis(benzyloxy)-5-((benzyloxy)methyl)tetrahydrofuran-2-yl)-4-fluoroisothiazole-3-carboxylate. A stirred mixture of tert-butyl 5-((2R,3R,4R,5R)-3,4-bis(benzyloxy)-5-((benzyloxy)methyl)tetrahydrofuran-2-yl)-4-(trimethylstannyl)isothiazole-3-carboxylate (320 mg, 426.37 μmol), Selectfluor® (302.09 mg, 852.73 μmol), NaHCO₃ (71.64 mg, 852.73 μmol, 33.16 μL), Ag₂O (10.56 mg, 85.27 μmol, 1.41 μL), AgOTf (131.46 mg, 511.64 μmol) in acetone (23 mL) in a sealed vessel was heated at 65° C. for 3 h. and shielded from light. The reaction mixture was filtered and the filtrate was concentrated under reduced pressure. The residue was purified by column chromatography (FCC, SiO₂, PE/EA=30/1 to 17/1) to give the title compound (60 mg, 23.23% yield, 100% purity) as colorless oil. LCMS: ESI-MS: m/z 628.1 [M+Na]⁺.

Step F: 5-((2R,3R,4R,5R)-3,4-Bis(benzyloxy)-5-((benzyloxy)methyl)tetrahydrofuran-2-yl)-4-fluoroisothiazole-3-carboxamide. A: tert-butyl 5-((2R,3R,4R,5R)-3,4-bis(benzyloxy)-5-((benzyloxy)methyl)tetrahydrofuran-2-yl)-4-fluoroisothiazole-3-carboxylate (42 mg, 69.34 μmol) was treated with NH₃-MeOH (10 mL). The mixture was stirred at 50° C. for 18 h. The reaction was set up for two batches. B: tert-butyl 5-((2R,3R,4R,5R)-3,4-bis(benzyloxy)-5-((benzyloxy)methyl)tetrahydrofuran-2-yl)-4-fluoroisothiazole-3-carboxylate (60 mg, 99.06 μmol) was treated with NH₃-MeOH (10 mL). The mixture was stirred at 50° C. for 18 h. The three above reaction mixtures were concentrated under reduced pressure. The residue was purified by column chromatography (SiO₂, PE/EA=20/1 to 3/1) to give the title compound (130 mg, 95.69% yield) as light yellow solid. ¹H NMR (400 MHz, CDCl₃) δ=7.36-7.28 (m, 15H), 6.92 (br, s, 1H), 5.57 (br, s, 1H), 5.41 (d, J=4.8 Hz, 1H), 4.63-4.61 (m, 2H), 4.60-4.58 (m, 1H), 4.56 (s, 1H), 4.54 (s, 1H), 4.51-4.49 (m, 1H), 4.37-4.32 (m, 1H), 4.09-4.06 (m, 1H), 4.03-3.98 (m, 1H), 3.69 (dd, J=3.0, 10.8 Hz, 1H), 3.57 (dd, J=3.5, 10.8 Hz, 1H).

Step G: 5-((2R,3R,4S,5R)-3,4-Dihydroxy-5-(hydroxymethyl)tetrahydrofuran-2-yl)-4-fluoroisothiazole-3-carboxamide. To a solution of 5-((2R,3R,4R,5R)-3,4-bis(benzyloxy)-5-((benzyloxy)methyl)tetrahydrofuran-2-yl)-4-fluoroisothiazole-3-carboxamide (130 mg, 236.96 μmol) in DCM (1 mL) was added BCl₃ (1 M, 2.37 mL, 10 eq.) at −78° C. under N2. The mixture was stirred at 0° C. for 2 h. The reaction mixture was quenched with MeOH (10 mL), and NH₃—H₂O (0.5 mL) and stirred for 1 h. The mixture was concentrated in vacuum. The residue was purified by column chromatography (FCC, SiO₂, DCM/MeOH=25/1 to 10/1) to give the title compound (44 mg, 65.00% yield, 97.4% purity) as white solid. ¹H NMR (400 MHz, CD₃OD) δ=5.14 (dd, J=0.9, 5.7 Hz, 1H), 4.13-4.09 (m, 1H), 4.07 (t, J=4.7 Hz, 1H), 4.01 (q, J=4.4 Hz, 1H), 3.79-3.72 (m, 1H), 3.70-3.64 (m, 1H). ¹⁹F NMR (376 MHz, CD₃OD) b 140.5. MS:ESI-MS: m/z 279.04 [M+H]⁺.

Step H. ((2R,3S,4R,5R)-5-(3-Carbamoyl-4-fluoroisothiazol-5-yl)-3,4-dihydroxytetrahydrofuran-2-yl)methyl tetrahydrogen triphosphate. The title compound was prepared in a manner analogous to Example 8. MS: m/z=517.1 (M−1)⁻. ³¹P-NMR (D₂O), δ=−11.03 (d), −11.67 (d), −23.52 (t).

### Example 13: ((2R,3S,4R,5S)-5-(2-Carbamoyloxazol-4-yl)-3,4-dihydroxytetrahydrofuran-2-yl)methyl tetrahydrogen triphosphate

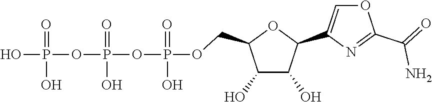 [SMILES: NC(=O)c1nc([C@@H]2O[C@H](COP(=O)(O)OP(=O)(O)OP(=O)(O)O)[C@@H](O)[C@H]2O)co1]

Step A: 4-((2S,3S,4R,5R)-3,4-Bis(benzyloxy)-5-((benzyloxy)methyl)tetrahydrofuran-2-yl)-2-vinyloxazole. To a solution of 1-((2R,3R,4R,5R)-3,4-bis(benzyloxy)-5-((benzyloxy)methyl)tetrahydrofuran-2-yl)-2-bromoethan-1-one (Intermediate 8, 3 g, 5.71 mmol) in EA (30 mL) was added s triflate (1.91 g, 7.42 mmol) and acrylamide (527.58 mg, 7.42 mmol). The mixture was stirred at 70° C. for 12 h. The reaction mixture was cooled down and filtered, and the filtrate was concentrated at low pressure. The residue was purified by column chromatography (FCC, SiO₂, Petroleum ether/Ethyl acetate=50/1 to 1:1) to give the title compound (1.2 g, 42.24% yield, 100% purity) was obtained as a colorless oil. ¹H NMR (400 MHz, CDCl₃) δ=7.50 (s, 1H), 7.41-7.12 (m, 15H), 6.56 (dd, J=11.2, 17.6 Hz, 1H), 6.14 (dd, J=0.9, 17.6 Hz, 1H), 5.61 (dd, J=0.9, 11.2 Hz, 1H), 5.04 (d, J=4.6 Hz, 1H), 4.67-4.59 (m, 3H), 4.59-4.45 (m, 3H), 4.31 (td, J=4.2, 6.0 Hz, 1H), 4.16 (t, J=4.9 Hz, 1H), 4.08-4.01 (m, 1H), 3.77-3.65 (m, 1H), 3.62-3.52 (m, 1H). LCMS: ESI-MS: m/z=520.1 [M+Na]⁺.

Step B: 1-(4-((2S,3S,4R,5R)-3,4-Bis(benzyloxy)-5-((benzyloxy)methyl)tetrahydrofuran-2-yl)oxazol-2-yl)ethane-1,2-diol. To a solution of 4-((2S,3S,4R,5R)-3,4-bis(benzyloxy)-5-((benzyloxy)methyl)tetrahydrofuran-2-yl)-2-vinyloxazole (1.2 g, 2.41 mmol) in THF (20 mL) and H₂O (2 mL) was added by OsO₄ (0.1 M in H₂O, 7.24 mL) and NMO (423.79 mg, 3.62 mmol, 381.79 μL). The mixture was stirred at 25° C. stirred for 12 h. The reaction mixture was quenched with saturated aq. Na₂SO₃ solution (20 mL), and extracted with EA (20 mL×2). The resulting solution was dried over anhydrous Na₂SO₄, and concentrated at low pressure. The residue was purified by column chromatography (FCC, SiO₂, Petroleum ether/Ethyl acetate=20/1 to 1:2) to give the title compound (0.860 g, 67.08% yield, 100% purity) as yellow oil. LCMS: ESI-MS: m/z=554.1 [M+Na]⁺

Step C: 4-((2S,3S,4R,5R)-3,4-Bis(benzyloxy)-5-((benzyloxy)methyl)tetrahydrofuran-2-yl)oxazole-2-carbaldehyde. To a solution of 1-(4-((2S,3S,4R,5R)-3,4-bis(benzyloxy)-5-((benzyloxy)methyl)tetrahydrofuran-2-yl)oxazol-2-yl)ethane-1,2-diol (600 mg, 1.13 mmol) in CH₃CN (5 mL) and H₂O (3 mL) was added by NalO₄ (724.24 mg, 3.39 mmol). The mixture was stirred at 25° C. for 1 h. The reaction mixture was diluted with EA (20 mL) and water (10 mL). The organic layer was washed with brine (10 mL) and dried over anhydrous Na₂SO₄, and concentrated at low pressure. The residue was purified by column chromatography (FCC, SiO₂, Petroleum ether/Ethyl acetate=20/1 to 1:2) to give the title compound (0.470 g, 83.36% yield) as yellow oil. LCMS: ESI-MS: m/z=522.0 [M+Na]⁺.

Step D: 4-((2S,3S,4R,5R)-3,4-Bis(benzyloxy)-5-((benzyloxy)methyl)tetrahydrofuran-2-yl)oxazole-2-carboxylic acid. To a solution of 4-((2S,3S,4R,5R)-3,4-bis(benzyloxy)-5-((benzyloxy)methyl)tetrahydrofuran-2-yl)oxazole-2-carbaldehyde (450 mg, 900.80 μmol) in t-BuOH (3 mL) and H₂O (2 mL) was added NaH₂PO₄ (108.08 mg, 900.80 μmol), 2-methyl-2-butene (277.97 mg, 3.96 mmol, 419.90 μL), and sodium chlorite (358.47 mg, 3.96 mmol). The mixture was stirred at 25° C. for 1 h. The reaction mixture was diluted with water (20 mL). The reaction mixture was extracted by EA (10 mL×2) and the organic layer was washed with brine (10 mL), dried over anhydrous Na₂SO₄, and concentrated at low pressure. The residue was purified by column chromatography (FCC, SiO₂, DCM/MeOH=100/1 to 8:1) give the title compound (0.32 g, 68.90% yield, 100% purity) as yellow oil. LCMS: ESI-MS: m/z=538.1 [M+Na]⁺.

Step E: 4-((2S,3S,4R,5R)-3,4-Bis(benzyloxy)-5-((benzyloxy)methyl)tetrahydrofuran-2-yl)oxazole-2-carboxamide. A mixture of 4-((2S,3S,4R,5R)-3,4-bis(benzyloxy)-5-((benzyloxy)methyl)tetrahydrofuran-2-yl)oxazole-2-carboxylic acid (200 mg, 360.78 mol), HATU (274.36 mg, 721.56 μmol) in DMF (3 mL) was stirred 15 min, and NH₃ (1 M, 1 mL in THF) was added and stirred at 25° C. for 2 h. The reaction mixture was diluted with water (5 mL). The reaction mixture was extracted by EA (5 mL×3). The organic layer was washed with (brine:H₂O=1:1, 5 mL×2), and the organic layer was dried over anhydrous Na₂SO₄, and concentrated at low pressure. The residue was purified by column chromatography (FCC, SiO₂, Petroleum ether/Ethyl acetate=5/1 to 1:1) to give the title compound (0.151 g, 81.34% yield) as yellow oil. ¹H NMR (400 MHz, CDCl₃) δ=7.65 (s, 1H), 7.41-7.20 (m, 15H), 6.63 (br s, 1H), 5.44 (br s, 1H), 5.03 (d, J=5.1 Hz, 1H), 4.70-4.58 (m, 3H), 4.58-4.48 (m, 3H), 4.37-4.29 (m, 1H), 4.19-4.10 (m, 1H), 4.10-4.05 (m, 1H), 3.73-3.65 (m, 1H), 3.65-3.53 (m, 1H). LCMS: ESI-MS: m/z=515.0 [M+H]⁺.

Step F: 4-((2S,3R,4S,5R)-3,4-Dihydroxy-5-(hydroxymethyl)tetrahydrofuran-2-yl)oxazole-2-carboxamide. To a solution of 4-((2S,3S,4R,5R)-3,4-bis(benzyloxy)-5-((benzyloxy)methyl)tetrahydrofuran-2-yl)oxazole-2-carboxamide (150 mg, 271.10 μmol) in DCM (1 mL) was added BCl₃ (1 M, 2.71 mL, 10 eq in DCM) at −78° C. and stirred at 0° C. for 2 h. The reaction mixture was quenched with MeOH (5 mL) and stirred for 30 min, and the reaction mixture was concentrated at low pressure. The residue was purified by column chromatography (FCC, SiO₂, DCM/MeOH=30/1 to 10:1) twice to give the title compound (48 mg, 72.07% yield, 99.4% purity) as yellow oil. ¹H NMR (400 MHz, CD₃OD) δ=8.07 (s, 1H), 4.78 (d, J=5.3 Hz, 1H), 4.24-4.15 (m, 1H), 4.11 (t, J=5.1 Hz, 1H), 3.98-3.92 (m, 1H), 3.80-3.74 (m, 1H), 3.68-3.61 (m, 1H) MS: ESI-MS: m/z=245.08[M+H]⁺.

Step G. ((2R,3S,4R,5S)-5-(2-Carbamoyloxazol-4-yl)-3,4-dihydroxytetrahydrofuran-2-yl)methyl tetrahydrogen triphosphate. The title compound was prepared in a manner analogous to Example 8. MS: m/z=483.2 (M−1)⁻. ³¹P-NMR (D₂O), δ=−8.14(d), −11.18 (d), −22.37 (t).

### Biological Assays

EC₅₀ [uM] as Measured Using Two Cell Lines: WSN/33 (H1N1) A549 and MDCK

1. Human lung carcinoma A549 cells (ATCC, Manassas, VA) were plated at a density of 5×10⁴ cells/ml (5×10³ cells/well) in maintenance media (Ham's F12 media supplemented with 10% FBS, 1% penicillin/streptomycin, 1% HEPES, 1% Glutamine and 1% non-essential amino acids (all Mediatech, Manassas, VA) in 96-well plates. After 24 hours, serially diluted compounds in assay media (Ham's F12 supplemented with 0.3FBS, 1% penicillin/streptomycin, 1% HEPES, 1% Glutamine and 1% non-essential amino acids) were added to cells and incubated for an additional 24 hours. Cells were infected with 250 IU/well of Influenza strains A/WSN/33 (H₁N1) (Virapur, San Diego CA) and incubated for 20 h at 37° C., 5% CO₂. The cell culture supernatant was aspirated off and 50 μl 25 μM 2′-(4-Methylumbelliferyl)-a-D-N-acetylneuraminic acid (MUNANA, Sigma-Aldrich) dissolved in 33 mM MES, pH 6.5 (Emerald Biosystems, Bainbridge Island, WA) was added to the cells. After incubation for 45 min at 30° C., reactions were stopped by addition of 150 μL stop solution (100 mM glycine, pH 10.5, 25% ethanol, all Sigma-Aldrich). Fluorescence was measured with excitation and emission filters of 355 and 460 nm, respectively, on a Victor X3 multi-label plate reader (Perkin Elmer, Waltham, MA). Cytotoxicity of uninfected parallel cultures was determined by addition of 100 μL CellTiter-Glo® reagent (Promega, Madison, WI), and incubation for 10 min at room temperature. Luminescence was measured on a Victor X3 multi-label plate reader.

2. Alternatively, Madin-Darby canine kidney epithelial cells (MDCK, ATCC), were plated at a density of 7.5×10⁴ cells/ml (7.5×10³ cells/well) in maintenance media (DMEM with same supplements as above) in 96-well plates. After 24 hours, serially diluted compounds in assay media (MEM supplemented with 0.3FBS, 1% penicillin/streptomycin, 1% HEPES, 1% Glutamine and 1% non-essential amino acids) were added to cells and incubated for an additional 24 hours. Cells were infected with 250 IU/well of Influenza strains A/WSN/33 (H1N1) and incubated for 20 h at 37° C., 5% CO₂. The cell culture supernatant was aspirated off and 50 μL 25 μM 2′-(4-Methylumbelliferyl)-a-D-N-acetyl neuraminic acid (MUNANA, Sigma-Aldrich) dissolved in 33 mM MES, pH 6.5 (Emerald Biosystems, Bainbridge Island, WA) was added to the cells. After incubation for 45 min at 30° C., reactions were stopped by addition of 150 μl stop solution (100 mM glycine, pH 10.5, 25% ethanol, all Sigma-Aldrich). Fluorescence was measured with excitation and emission filters of 355 and 460 nm, respectively, on a Victor X3 multi-label plate reader (Perkin Elmer, Waltham, MA). Cytotoxicity of uninfected parallel cultures was determined by addition of 100 μL CellTiter-Glo® reagent (Promega, Madison, WI), and incubation for 10 min at room temperature. Luminescence was measured on a Victor X3 multi-label plate reader.

| | EC₅₀ [uM] | | |
|---|---|---|---|
| | WSN/33 | EC₅₀ [uM] WSN/33 | |
| Example # | (H1N1) A549 | (H1N1) MDCK | Structure |
| 1 | 1.35 | n.d. | 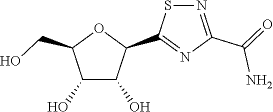 [SMILES: NC(=O)c1nsc([C@@H]2O[C@H](CO)[C@@H](O)[C@H]2O)n1] |
| 2 | 54.7 | 0.32 | 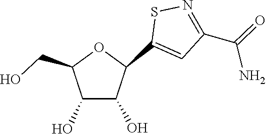 [SMILES: NC(=O)c1cc([C@@H]2O[C@H](CO)[C@@H](O)[C@H]2O)sn1] |
| 3 | 40.7 | 10.1 | 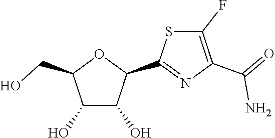 [SMILES: NC(=O)c1nc([C@@H]2O[C@H](CO)[C@@H](O)[C@H]2O)sc1F] |
| 4 | 15.1 | n.d. | 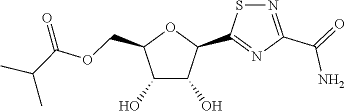 [SMILES: CC(C)C(=O)OC[C@H]1O[C@@H](c2nc(C(N)=O)ns2)[C@H](O)[C@@H]1O] |
| 6 | 4.66 | 1.85 | 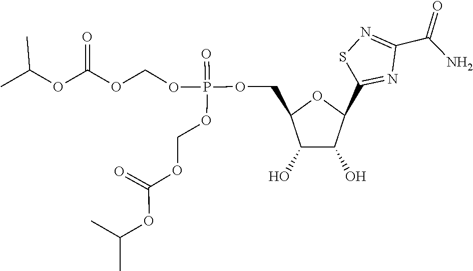 [SMILES: CC(C)OC(=O)OCOP(=O)(OCOC(=O)OC(C)C)OC[C@H]1O[C@@H](c2nc(C(N)=O)ns2)[C@H](O)[C@@H]1O] |
| 7 | 1.35 | n.d. | 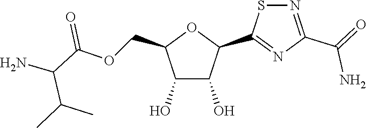 [SMILES: CC(C)C(N)C(=O)OC[C@H]1O[C@@H](c2nc(C(N)=O)ns2)[C@H](O)[C@@H]1O] |

n.d. means not determined

IC₅₀ [uM] IAVpol (Nanchang/H3N2)

### Influenza Polymerase Assay and Compound IC₅₀ Measurement

The nucleotide incorporation activity of IAV PA/PB1/PB2 complex (from the H₃N2 IAV strain (A/chicken/Nanchang/3-120/01) is measured as an incorporation of tritiated UMP into acid-insoluble RNA products. The reactions contain 30 nM recombinant enzyme, 100 nM IAV mini-genome RNA, 0.5 μM 5′vRNA, 100 μM ATP, 100 μM GTP, 100 μM CTP, 0.5 μM tritiated UTP, 40 mM Tris-HCl (pH 7.4), 0.4 U/□L RNaseIn, 0.2 mg/mL BSA, 50 mM NaCl, 2 mM dithiothreitol, 5 mM MgCl₂. Standard reactions are incubated for 2 hours at 37° C., in the presence of increasing concentration of inhibitor. At the end of the reaction, RNA is precipitated with 10% TCA, and acid-insoluble RNA products are filtered on a size exclusion 96-well plate. After washing of the plate, scintillation liquid is added and radiolabeled RNA products are detected according to standard procedures with a Trilux Topcount scintillation counter. The compound concentration at which the enzyme-catalyzed product formation rate is reduced by 50% (IC₅₀) is calculated by non-linear regression data fitting to a sigmoidal dose-response equation.

| | IC₅₀ [uM] IAVpol | |
|---|---|---|
| Example # | (Nanchang/H3N2) | Structure |
| 9 | 2.49 | 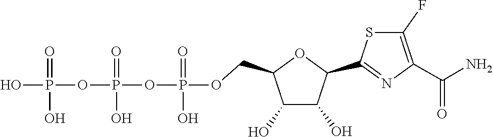 [SMILES: NC(=O)c1nc([C@@H]2O[C@H](COP(=O)(O)OP(=O)(O)OP(=O)(O)O)[C@@H](O)[C@H]2O)sc1F] |
| 10 | 1.11 | 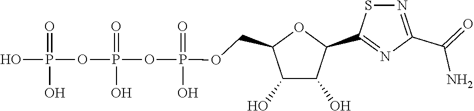 [SMILES: NC(=O)c1nsc([C@@H]2O[C@H](COP(=O)(O)OP(=O)(O)OP(=O)(O)O)[C@@H](O)[C@H]2O)n1] |
| 11 | 18.7 | 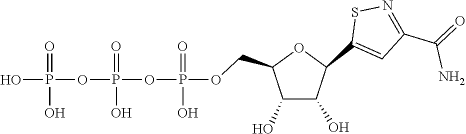 [SMILES: NC(=O)c1cc([C@@H]2O[C@H](COP(=O)(O)OP(=O)(O)OP(=O)(O)O)[C@@H](O)[C@H]2O)sn1] |
| 12 | 7.31 | 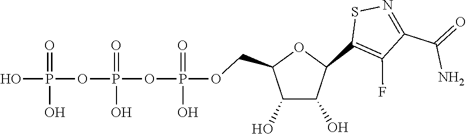 [SMILES: NC(=O)c1nsc([C@@H]2O[C@H](COP(=O)(O)OP(=O)(O)OP(=O)(O)O)[C@@H](O)[C@H]2O)c1F] |
| 13 | 10.0 | 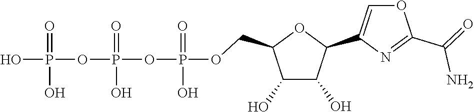 [SMILES: NC(=O)c1nc([C@@H]2O[C@H](COP(=O)(O)OP(=O)(O)OP(=O)(O)O)[C@@H](O)[C@H]2O)co1] |

### RSV Subgenomic Replicon

The RSV subgenomic replicon 395 Hela was licensed from Apath (Brooklyn, NY) and was originally developed by Dr. Mark Meeples of Center for Vaccines & Immunity, the Research Institute at Nationwide Children's Hospital in Columbus, Ohio [2]. In brief, to generate subgenomic RSV replicon, three glycoprotein genes, those for SH, G, and F, from a full-length recombinant GFP-expressing (rg) RSV antigenomic cDNA were deleted. In their place, a blasticidin S deaminase (bsd) gene was inserted. Through multiple steps, the RSV replicon was established in Hela cells. The 395 Hela cells were cultured in Dulbecco's Modified Eagle Medium (DMEM) containing 4500 mg/L D-glucose, L-glutamine, and 110 mg/L sodium pyruvate (Invitrogen, Cat. #11995-040). The medium was further supplemented with 10% (v/v) fetal bovine serum (FBS) (Mediatech, Cat. #35-010-CV), 1% (v/v) penicillin/streptomycin (Mediatech, Cat. #30-002-CI), and 10 μg/mL of Blasticidin (BSD) (Invivogen, Cat. #ant-bl-1). Cells were maintained at 37° C. in a humidified 5% CO₂ atmosphere.

### Drug Treatment

Determination of 50% inhibitory concentration (IC₅₀), 90% inhibitory concentration (IC₉₀) and 50% cytotoxic concentration (CC₅₀) in RSV replicon cells were performed by the following procedure. On the first day, 5000 RSV replicon cells per well were plated in a 96-well plate. On the following day, the propagation medium was removed and replaced with cell media containing the following percentages of human serum: 5%, 10%, 20%, and 40% (v/v) along with the appropriate concentrations of penicillin/streptomycin, BSD. The compounds to be tested were solubilized in 100% DMSO to 100×the desired final testing concentration. Each compound was serially diluted (1:3) up to 9 distinct concentrations. Compounds in 100% DMSO were reduced to 10% (v/v) DMSO by diluting 1:10 in cell culture media. A 10 μL sample of the compounds diluted to 10% (v/v) DMSO with cell culture media was used to treat the RSV replicon cells in 96-well format. The final DMSO concentration was 1% (v/v). Cells were incubated with compounds for 7 days at 37° C. in a 5% CO₂ atmosphere. In each assay, positive control that was previously characterized in RSV replicon assay was included.

### Determination of Anti-Activity

### Renilla Luciferase Assay

The Renilla Luciferase Assay System (Promega, Cat. #E2820) was used to measure anti-RSV replicon activity. Assay plates were set up as stated above (see Section 4.3). Luminescence was recorded using a Perkin Elmer multilabel counter Victor3V. IC₅₀, the concentration of the drug required for reducing RSV replicon RNA by 50% in relation to the untreated cell control value, was calculated from the plot of percentage reductions of the optical density (OD) value against the drug concentrations using the Microsoft Excel forecast function.

### Cell Viability Assay

395 Hela cell proliferation assay (Promega, CellTiter-Glo Luminescent Cell Viability Assay, Cat. #G7572) was used to measure cell viability. The CellTiter-Glo® Luminescent Cell Viability Assay is a homogeneous method to determine the number of viable cells in culture based on quantitation of the ATP present, which signals the presence of metabolically active cells. Assay plates were set up in the same format as in the replicon assay (see Section 4.4). CellTiter-Glo reagent (100 μL) was added to each well and incubated at room temperature for 8 minutes. Luminescence was recorded using a Perkin Elmer multilabel counter Victor3V. The CC₅₀, the concentration of the drug required for reducing viable cells by 50% in relation to the untreated cell control value, was calculated from the plot of percentage reductions of the luminescence value against the drug concentrations using the Microsoft Excel forecast function.

What is claimed:

1. A compound, and pharmaceutically acceptable salts, solvates, stereoisomers, isotopic variants, or N-oxides thereof, having the structure of Formula (I): 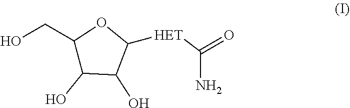
wherein
HET is a heteroaryl selected from the group consisting of:

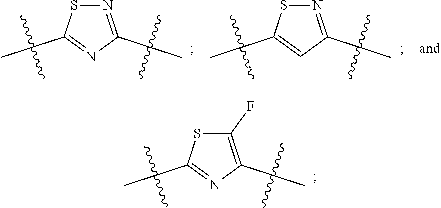 [SMILES: *c1nsc(*)n1 ; *c1cc(*)sn1 ; *c1nc(*)c(F)s1]

2. The compound of claim 1, wherein HET is

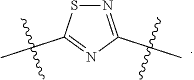 [SMILES: *c1nsc(*)n1]

3. The compound of claim 1, wherein HET is

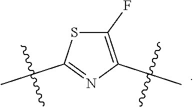 [SMILES: *c1nc(*)c(F)s1]

4. The compound of claim 1, wherein HET is

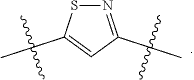 [SMILES: *c1cc(*)sn1]

5. A compound selected from the group consisting of: 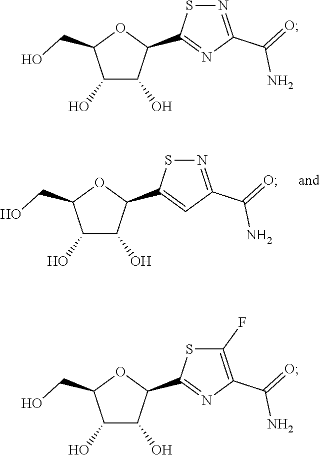 [SMILES: NC(=O)c1nsc([C@@H]2O[C@H](CO)[C@@H](O)[C@H]2O)n1 ; NC(=O)c1cc([C@@H]2O[C@H](CO)[C@@H](O)[C@H]2O)sn1 ; "and" ; NC(=O)c1nc([C@@H]2O[C@H](CO)[C@@H](O)[C@H]2O)sc1F]
and pharmaceutically acceptable salts, solvates, or N-oxides thereof.

6. A compound of claim 5, wherein the compound is: 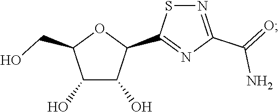 [SMILES: NC(=O)c1nsc([C@@H]2O[C@H](CO)[C@@H](O)[C@H]2O)n1]
and pharmaceutically acceptable salts, solvates, or N-oxides thereof.

7. A compound, and pharmaceutically acceptable salts, solvates, stereoisomers, isotopic variants, or N-oxides thereof, having the structure of Formula (II): 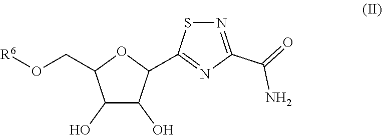
wherein
R⁶ is —(C═O)C₁₋₆alkyl, or —(C═O)C₁₋₆alkyl wherein C₁₋₆alkyl is substituted with NH₂.

8. A compound of claim 7, selected from the group consisting of: 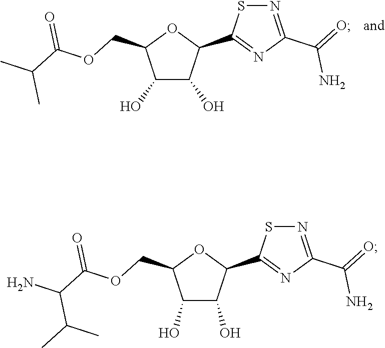 [SMILES: CC(C)C(=O)OC[C@H]1O[C@@H](c2nc(C(N)=O)ns2)[C@H](O)[C@@H]1O ; CC(C)C(N)C(=O)OC[C@H]1O[C@@H](c2nc(C(N)=O)ns2)[C@H](O)[C@@H]1O]
and pharmaceutically acceptable salts, solvates, or N-oxides thereof.

9. A compound, and pharmaceutically acceptable salts, solvates, stereoisomers, isotopic variants, or N-oxides thereof, having the structure of Formula (III): 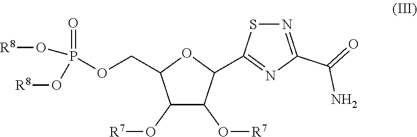
wherein
R⁷ is H or two R⁷ members come together to form a 5-membered ring substituted with OCH₃; and
R⁸ is —CH₂O—(C═O)—O—C₁₋₆alkyl.

10. A compound selected from: 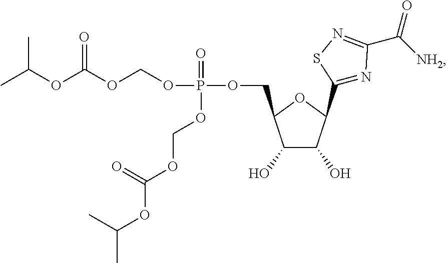 [SMILES: CC(C)OC(=O)OCOP(=O)(OCOC(=O)OC(C)C)OC[C@H]1O[C@@H](c2nc(C(N)=O)ns2)[C@H](O)[C@@H]1O]
and pharmaceutically acceptable salts, solvates, or N-oxides thereof.

11. A compound, and pharmaceutically acceptable salts, solvates, stereoisomers, isotopic variants, or N-oxides thereof, having the structure of Formula (IV): 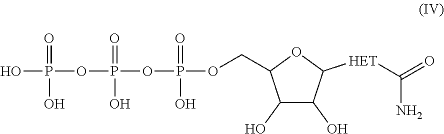
wherein
HET is a heteroaryl selected from the group consisting of:

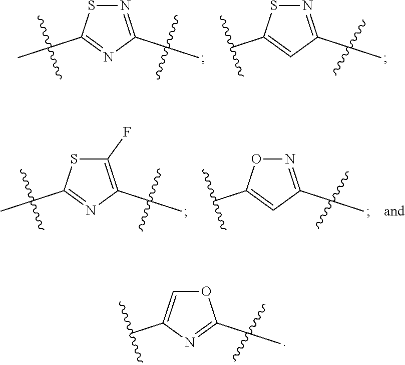 [SMILES: *c1nsc(*)n1 ; *c1cc(*)sn1 ; *c1nc(*)c(F)s1 ; *c1cc(*)on1 ; *c1coc(*)n1]

12. A compound of claim 11, selected from the group consisting of: 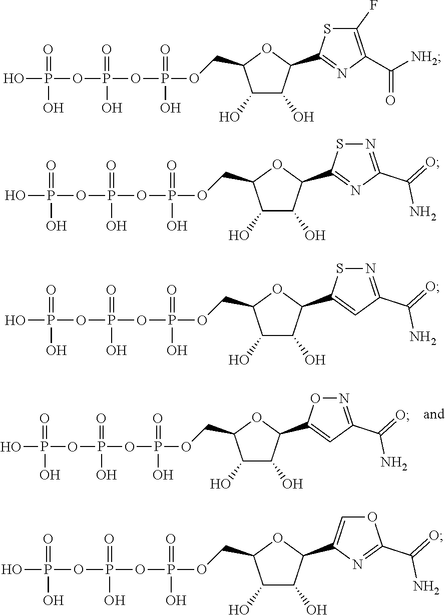
and pharmaceutically acceptable salts, solvates, or N-oxides thereof.

13. A pharmaceutical composition comprising:
(A) an effective amount of at least one compound selected from compounds of Formula (I) wherein: 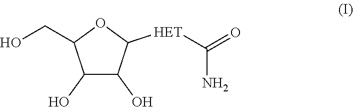
wherein
HET is a heteroaryl selected from the group consisting of: 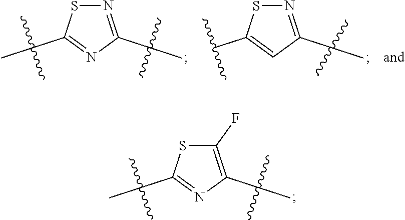 [SMILES: *c1nsc(*)n1 ; *c1cc(*)sn1 ; *c1nc(*)c(F)s1]
and pharmaceutically acceptable salts, solvates, stereoisomers, isotopic variants, or N-oxides of compounds of Formula (I); and
(B) at least one pharmaceutically acceptable excipient.

14. A pharmaceutical composition comprising an effective amount of at least one compound of claim 5 and at least one pharmaceutically acceptable excipient.

15. A pharmaceutical composition comprising an effective amount of a compound of claim 6 and at least one pharmaceutically acceptable excipient.

16. A pharmaceutical composition comprising an effective amount of at least one compound of claim 8 and at least one pharmaceutically acceptable excipient.

17. A pharmaceutical composition comprising an effective amount of at least one compound of claim 10 and at least one pharmaceutically acceptable excipient.

18. A pharmaceutical composition comprising an effective amount of at least one compound of claim 12 and at least one pharmaceutically acceptable excipient.

19. A method of treating an orthomyxovirus infection in a subject, said method comprising administering to the subject in need of such treatment an effective amount of at least one compound selected from compounds of Formula (I): 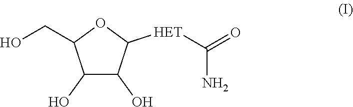
wherein
HET is a heteroaryl selected from the group consisting of: 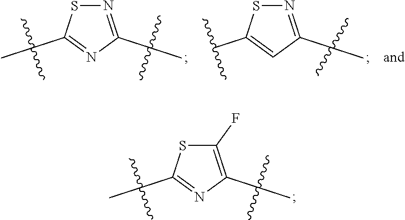 [SMILES: *c1nsc(*)n1 ; *c1cc(*)sn1 ; *c1nc(*)c(F)s1]
and pharmaceutically acceptable salts, solvates, stereoisomers, isotopic variants, or N-oxides of compounds of Formula (I).

20. The method of claim 19, wherein the orthomyxovirus is influenza.

21. The method of claim 20, wherein the influenza is influenza A.

22. The method of claim 21, wherein the influenza is influenza A subtype H3N2.

23. The method of claim 21, wherein the influenza A is of a strain resistant to amantadine, rimantadine, or oseltamivir.

24. The method of claim 20, wherein the influenza is influenza B.

25. The method of claim 19, wherein the compound is administered orally.

26. A method of treating an orthomyxovirus infection in a subject, said method comprising administering to the subject in need of such treatment an effective amount of 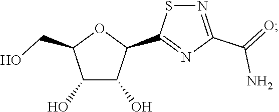 [SMILES: NC(=O)c1nsc([C@@H]2O[C@H](CO)[C@@H](O)[C@H]2O)n1]
and pharmaceutically acceptable salts, solvates, stereoisomers, isotopic variants, or N-oxides thereof.

27. A method of treating an orthomyxovirus infection in a subject, said method comprising administering to the subject in need of such treatment an effective amount of a pharmaceutical composition comprising:
(A) an effective amount of at least one compound selected from compounds of Formula (I) wherein: 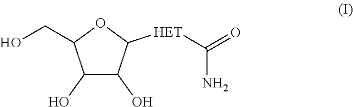
wherein
HET is a heteroaryl selected from the group consisting of: 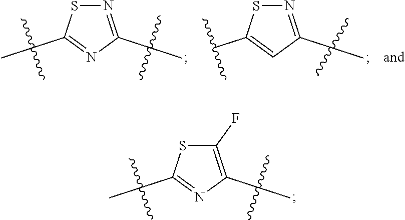 [SMILES: *c1nsc(*)n1 ; *c1cc(*)sn1 ; *c1nc(*)c(F)s1]
and pharmaceutically acceptable salts, solvates, stereoisomers, isotopic variants, or N-oxides of compounds of Formula (I); and
(B) at least one pharmaceutically acceptable excipient.

28. A method of treating an orthomyxovirus infection in a subject, said method comprising administering to the subject in need of such treatment an effective amount of a pharmaceutical composition comprising an effective amount of at least one compound of claim 5 and at least one pharmaceutically acceptable excipient

29. A method of treating an orthomyxovirus infection in a subject, said method comprising administering to the subject in need of such treatment an effective amount of a pharmaceutical composition comprising an effective amount of at least one compound of claim 6 and at least one pharmaceutically acceptable excipient

30. A method of treating an orthomyxovirus infection in a subject, said method comprising administering to the subject in need of such treatment an effective amount of a pharmaceutical composition comprising an effective amount of at least one compound of claim 8 and at least one pharmaceutically acceptable excipient

31. A method of treating an orthomyxovirus infection in a subject, said method comprising administering to the subject in need of such treatment an effective amount of a pharmaceutical composition comprising an effective amount of at least one compound of claim 10 and at least one pharmaceutically acceptable excipient

32. A method of treating an orthomyxovirus infection in a subject, said method comprising administering to the subject in need of such treatment an effective amount of a pharmaceutical composition comprising an effective amount of at least one compound of claim 12 and at least one pharmaceutically acceptable excipient
